(19) United States
(12) Patent Application Publication
(10) Pub. No.: US 2026/0207632 A1
(43) Pub. Date: Jul. 23, 2026

(54) CANNABINOID BASED THERAPY

(71) Applicant: KINGDOM THERAPEUTICS LIMITED, Wells (GB)

(72) Inventors: Kathleen FADDEN, Wells (GB); Liz SHANAHAN, Wells (GB)

(21) Appl. No.: 19/139,330

(22) PCT Filed: Dec. 14, 2023
(86) PCT No.: PCT/GB2023/053239

§ 371 Date: Jun. 14, 2025

(30) Foreign Application Priority Data
Dec. 16, 2022 (GB) 2219039.1
Oct. 18, 2023 (GB) 2315908.0

(51) Int. Cl. A61K 31/00; A61P 1/00; A61P 25/00

(52) U.S. Cl. CPC A61K 31/658; A61P 1/00; A61P 25/00

(57) ABSTRACT

This invention relates to a cannabinoid for use in the prevention, amelioration or treatment of autism spectrum disorder or a symptom thereof. The cannabinoid may prevent or reduce gastrointestinal/gut hyperpermeability, and may prevent or reduce colonic muscle thinning or increase colonic muscle thickness.

### FIELD OF THE INVENTION

This invention relates to a cannabinoid for use in the prevention, amelioration or treatment of autism spectrum disorder or a symptom thereof. The cannabinoid may prevent or reduce gastrointestinal/gut hyperpermeability. The cannabinoid may prevent or reduce colonic muscle thinning or increase colonic muscle thickness. The present invention also relates to a cannabinoid for use in the prevention, amelioration or treatment of gastrointestinal inflammation.

### BACKGROUND

ASD is a neurodevelopment disorder, where severe behavioural disorders emerge in early life and are characterised by issues with social interaction, communication, irritability, compulsive behaviours and in many cases, poor cognitive development. 40% of children with ASD are nonverbal and in addition to their neurological and behavioural symptoms, most suffer from persistent gastrointestinal-related issues. Today, it is estimated that more than 1 million children in the US and 1.3 million in the EU have ASD, with an incidence rate of 1 in 44, which is increasing. It is four times more common in boys than girls. It is recognised that patients suffering from ASD exhibit not only neurological and behavioural symptoms, but also gastrointestinal-related problems, including gut dysbiosis. The reason behind gastrointestinal disorders in ASD has not been fully elucidated, but many possible causes have been put forward: including an underlying genetic abnormality, impaired gut dysbiosis for example as described in US2020/0061127; picky eating, stress and an impaired stress response, and increased sensitivity to sensory input.

ASD is an area of significant unmet need. Currently, the primary drug prescribed in ASD is an antipsychotic, Risperidone™, which is licenced to treat irritability. As the underlying cause of many ASD symptoms remains unknown, individuals suffering from ASD have limited options for treating or managing their condition.

The endocannabinoid (eCB) system includes the CB1 cannabinoid receptor (CB1R), its endogenous ligands including the eCB anandamide (AEA), and the enzymes responsible for eCB synthesis and degradation. Endocannabinoid system signaling is a critical regulator of both glutamatergic and GABAergic synapses throughout the brain; thus, it is not surprising that alterations in AEA signaling have been demonstrated in multiple neurological and psychiatric disorders. Studies in animals and humans indicate a role for cannabinoid signaling in the regulation of social behavior, and eCB dysfunction has emerged as a common feature of multiple ASD animal models. In preclinical studies, cannabinoids have been shown to rescue social deficits and reduce repetitive behaviors in several different rodent models.

Cannabinoids, or agents which modulate the endocannabinoid system, have been proposed for treatment of ASD, or a symptom thereof. However, there remains a need for improved treatment of ASD or a symptom thereof, and gastrointestinal disorders.

### BRIEF SUMMARY OF THE DISCLOSURE

In accordance with a first aspect of the present invention, there is provided a cannabinoid for use in the prevention or treatment of gastrointestinal hyperpermeability in a subject.

Also provided is a method of preventing or treating gastrointestinal hyperpermeability in a subject, wherein the method comprises administering a cannabinoid to the subject.

In accordance with a second aspect of the present invention, there is provided a cannabinoid for use in the prevention or treatment of a symptom of ASD in a subject, wherein the subject has, or has been tested as having, gastrointestinal hyperpermeability.

Also provided is a method of preventing or treating a symptom of ASD in a subject, wherein the method comprises administering a cannabinoid to the subject, wherein the subject has, or has been tested as having, gastrointestinal hyperpermeability.

In accordance with a third aspect of the present invention, there is provided a cannabinoid for use in a method of preventing or treating gastrointestinal inflammation in a subject.

Also provided is a method of preventing or treating gastrointestinal inflammation in a subject, wherein the method comprises administering a cannabinoid to the subject.

In accordance with a fourth aspect of the present invention, there is provided a cannabinoid for use in the prevention or treatment of a symptom of ASD in a subject, by preventing or reducing gastrointestinal hyperpermeability.

Also provided is a method of preventing or treating a symptom of ASD in a subject, wherein the method comprises administering a cannabinoid to the subject, wherein the cannabinoid prevents or reduces gastrointestinal hyperpermeability.

In accordance with a fifth aspect of the present invention, there is provided a cannabinoid for use in the prevention or reduction in gastrointestinal inflammation in a subject, by preventing or reducing gastrointestinal hyperpermeability.

Also provided is a method of preventing or treating gastrointestinal inflammation in a subject, wherein the method comprises administering a cannabinoid to the subject, wherein the cannabinoid prevents or reduces gastrointestinal hyperpermeability.

In accordance with a sixth aspect of the present invention, there is provided a cannabinoid for use in the prevention or treatment of a symptom of ASD in a subject, by preventing or reducing colonic muscle thinning.

Also provided is a method of preventing or treating a symptom of ASD in a subject, wherein the method comprises administering a cannabinoid to the subject, wherein the cannabinoid prevents or reduces colonic muscle thinning.

In accordance with a seventh aspect of the present invention, there is provided a cannabinoid for use in the prevention or treatment of a symptom of ASD in a subject, wherein the subject has, or has been tested as having, colonic muscle thinning.

Also provided is a method of preventing or treating a symptom of ASD in a subject, wherein the method comprises administering a cannabinoid to the subject, wherein the subject has, or has been tested as having, gastrointestinal hyperpermeability.

### BRIEF DESCRIPTION OF THE DRAWINGS

Embodiments of the invention are further described hereinafter with reference to the accompanying drawings, in which:

FIG. 1 shows the VPA experimental timeline for the efficacy and combination study as described in the Examples.

FIG. 2 shows the effect of CBD (10, 30 and 60 mg/kg [Human Equivalent Dose (HED) 1.6, 4.8 & 9.6 mg/kg]) following 8 days drug treatment on social deficit following in utero valproic acid exposure. (A) latency, (B) frequency and (C) duration of social play in P30 animals. Data represents mean±SEM values during a 15-minute trial (n=4 pairs). *=P<0.05 compared to VEH/VEH; ##=P<0.01 compared to VPA/VEH.

FIG. 3 shows the effect of CBD (10, 30 and 60 mg/kg [HED 1.6, 4.8 & 9.6 mg/kg]) following 8 days drug treatment on social deficit following in utero valproic acid exposure. (A) latency, (B) frequency and (C) duration of play mount behaviour in P30 animals. Data represents mean±SEM values during a 15-minute trial (n=4 pairs). *−***=P<0.05−0.001 compared to VEH/VEH; #−###=P<0.05−0.001 compared to VPA/VEH.

FIG. 4 shows the effect of CBD (10, 30 and 60 mg/kg [HED 1.6, 4.8 & 9.6 mg/kg]) following 8 days drug treatment on social deficit following in utero valproic acid exposure. (A) latency, (B) frequency and (C) duration of pinning play behaviour in P30 animals. Data represents mean±SEM values during a 15-minute trial (n=4 pairs).

FIG. 5 shows the effect of CBD (10, 30 and 60 mg/kg; [HED 1.6, 4.8 & 9.6 mg/kg]) following 8 days drug treatment on irritability following in utero valproic acid exposure-effect on bottle brush reactivity in postnatal day 30 animals. Data represents mean±SEM (A) number of aggressive behaviours, (B) number of defensive behaviours and (C) irritability score (sum of aggressive and defensive behaviours) over ten 10-second trials (n=8). *−**=P<0.05−0.01 compared to VEH/VEH; #−####=P<0.05−0.0001 compared to VPA/VEH.

FIG. 6 shows the effect of CBD (10, 30 and 60 mg/kg; [HED 1.6, 4.8 & 9.6 mg/kg]) following 9 and 10 days drug treatment on open field locomotor activity (LMA) following in utero valproic acid exposure. Data represents mean±SEM of total distance travelled (cm) in the open field during two 10-minute trials on Day 1 (A) spontaneous LMA and Day 2 (B) habituated LMA (n=8). *−**=P<0.05−0.01 compared to VEH/VEH; #−###=P<0.05−0.001 compared to VPA/VEH.

FIG. 7 shows the effect of CBD (10, 30 and 60 mg/kg) following 9 and 10 days drug treatment on self-grooming and circling behaviour in the open field following in utero valproic acid exposure. Data represents mean±SEM of (A) Day 1 self-grooming and circling behaviour; (B) Day 2 self-grooming and circling behaviour (n=8). *−**=P<0.05−0.01 compared to VEH/VEH; ##−###=P<0.01-0.001 compared to VPA/VEH.

FIG. 8 shows the effect of CBD (10, 30 and 60 mg/kg; [HED 1.6, 4.8 & 9.6 mg/kg]) following 11 days drug treatment on gastrointestinal permeability following in utero valproic acid exposure. Data represents mean±SEM percent control fluorescence (530 nm) of FITC-labelled dextran in plasma collected four hours post oral gavage (n=8). ****=P<0.0001 compared to VEH/VEH; #−##=P<0.05−0.01 compared to VPA/VEH

FIG. 9 shows the effect of CBD (10, 30 and 60 mg/kg; [HED 1.6, 4.8 & 9.6 mg/kg]) following 12 days drug treatment on colonic myeloperoxidase (MPO) activity following in utero valproic acid exposure. Data represents mean±SEM percent control MPO activity (milliunits/mg protein) (n=8). ****=P<0.0001 compared to VEH/VEH; ###=P<0.001-compared to VPA/VEH.

FIG. 10 shows the effect of CBD alone (30 and 60 mg/kg; [HED 4.8 & 9.6 mg/kg]) and in combination with THC (0.75, 1.5 and 5 mg/kg; [0.12, 0.24 & 0.8 mg/kg HED]) following 8 days drug treatment on social deficit following in utero valproic acid exposure. (A) latency, (B) frequency and (C) duration of social play in P30 animals. Data represents mean±SEM values during a 15-minute trial (n=4 pairs). **=P<0.01 compared to VEH/VEH; #−##=P<0.05−0.01 compared to VPA/VEH.

FIG. 11 shows the effect of CBD alone (30 and 60 mg/kg; [HED 4.8 & 9.6 mg/kg]) and in combination with THC (0.75, 1.5 and 5 mg/kg) [0.12, 0.24 & 0.8 mg/kg HED] following 8 days drug treatment on social deficit following in utero valproic acid exposure. (A) latency, (B) frequency and (C) duration of pinning play behaviour in P30 animals. Data represents mean±SEM values during a 15-minute trial (n=4 pairs).

FIG. 12 shows the effect of CBD alone (30 and 60 mg/kg; [HED 4.8 & 9.6 mg/kg]) and in 5 combination with THC (0.75, 1.5 and 5 mg/kg) [0.12, 0.24 & 0.8 mg/kg HED] following 8 days drug treatment on irritability following in utero valproic acid exposure-effect on bottle brush reactivity in postnatal day 30 animals. Data represents mean±SEM (A) number of aggressive behaviours, (B) number of defensive behaviours and (C) irritability score (sum of aggressive and defensive behaviours) over ten 10-second trials (n=8). ***=P<0.001 compared to VEH/VEH; ###−####=P<0.001-0.0001 compared to VPA/VEH.

FIG. 13 shows the effect of CBD alone (30 and 60 mg/kg; [HED 4.8 & 9.6 mg/kg]) and in combination with THC (0.75, 1.5 and 5 mg/kg) [0.12, 0.24 & 0.8 mg/kg HED] following 9 and 10 days drug treatment on open field locomotor activity (LMA) following in utero valproic acid exposure. Data represents mean±SEM of total distance travelled (cm) in the open field during two 10-minute trials on Day 1 (A) spontaneous LMA and Day 2 (B) habituated LMA (n=8). ***−****=P<0.001-0.0001 compared to VEH/VEH; #−##=P<0.05−0.01 compared to VPA/VEH.

FIG. 14 shows the effect of CBD alone (30 and 60 mg/kg; [HED 4.8 & 9.6 mg/kg]) and in combination with THC (0.75, 1.5 and 3 mg/kg [HED 0.12, 0.24 & 0.47 mg/kg]) following 9 and 10 days drug treatment on self-grooming and circling behaviour in the open field following in utero valproic acid exposure. Data represents mean±SEM of (A) Day 1 self-grooming and circling behaviour; (B) Day 2 self-grooming and circling behaviour (n=8). *−**=P<0.05−0.01 compared to VEH/VEH; #−##=P<0.05−0.01 compared to VPA/VEH.

FIG. 15 shows the effect of CBD alone (30 and 60 mg/kg; [HED 4.8 & 9.6 mg/kg]) and in combination with THC (0.75, 1.5 and 3 mg/kg [HED 0.12, 0.24 & 0.47 mg/kg]) following 11 days drug treatment on gastrointestinal permeability following in utero valproic acid exposure. Data represents mean±SEM percent control fluorescence (530 nm) of FITC-labelled dextran in plasma collected four hours post oral gavage (n=8). ****=P<0.0001 compared to VEH/VEH; #−##=P<0.05−0.01 compared to VPA/VEH. 30 FIG. 16 shows the effect of CBD alone (30 and 60 mg/kg; [HED 4.8 & 9.6 mg/kg]) and in combination with THC (0.75, 1.5 and 3 mg/kg [HED 0.12, 0.24 & 0.47 mg/kg]) following 12 days drug treatment on colonic myeloperoxidase (MPO) activity following in utero valproic acid exposure. Data represents mean±SEM percent control MPO activity (milliunits/mg protein) (n=8). ****=P<0.0001 compared to VEH/VEH; ####=P<0.0001-compared to VPA/VEH.

FIG. 17 shows a comparison of colonic muscle thickness measurements in colonic tissue samples from test groups 1 to 5. Data were not normally distributed (as assessed by the Shapiro-Wilk test), thus statistical analysis was performed with a Kruskal-Wallis test followed by an uncorrected Dunn's post hoc test. Comparisons were considered significantly different if p<0.05. Data are presented as mean±SEM. A total of 125 measurements were taken from 5 animals for each treatment condition.

FIG. 18 shows microscope images of colonic tissue sections after hematoxylin and eosin staining. Each panel represents a colonic tissue section from one test group imaged at a magnification of 1.25×.

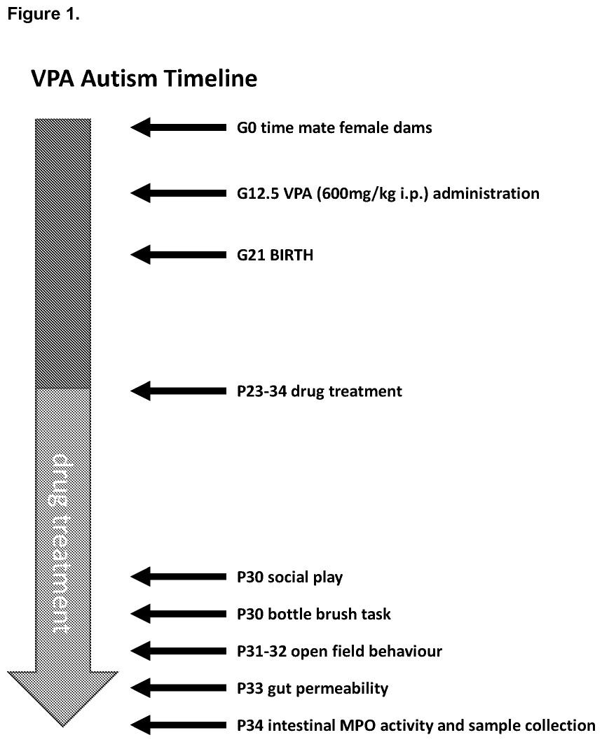

### DETAILED DESCRIPTION

The present invention is based upon the surprising finding by the present inventors that administration of a cannabinoid has the effect of reducing gastrointestinal hyperpermeability in a subject. There is a growing theory that many subjects, in particular children, with ASD who have gastrointestinal issues, may have leaky guts, or gastrointestinal hyperpermeability. When the gastrointestinal barrier in these subjects is compromised, metabolites in their intestines may leak into the blood stream and make their way to the brain. These subjects may have inflammation in their guts, and there is a growing belief that this not just exaggerates symptoms, but may even be causative. Gastrointestinal hyperpermeability has been associated with a range of conditions including gastrointestinal inflammation, behavioural symptoms and gastrointestinal disorders in ASD patients, and gastrointestinal conditions such as Crohn's disease or celiac disease, diabetes type 1, and arthritis in non-ASD patients. The present inventors have shown that a cannabinoid has the beneficial effect of reducing gastrointestinal hyperpermeability in a subject. The present inventors have shown that administration of a cannabinoid has the beneficial effect of reducing inflammation, and also improving ASD symptoms in a rat model of ASD. The present inventors have also shown that administration of a cannabinoid has the beneficial effect of increasing colonic muscle thickness.

The present invention in a first aspect therefore provides a cannabinoid for use in the prevention or treatment of gastrointestinal hyperpermeability in a subject. Also provided is a method of preventing or treating gastrointestinal hyperpermeability in a subject, wherein the method comprises administering a cannabinoid to the subject.

Suitably, the cannabinoid may prevent or reduce gastrointestinal hyperpermeability by reducing or preventing colonic muscle thinning, or increasing colonic muscle thickness, and/or maintaining colonic muscle thickness.

Suitably, the first aspect of the invention is useful in the prevention or treatment of a gastrointestinal symptom or disorder and/or ASD or a symptom thereof, suitably a gastrointestinal symptom of ASD, and/or a behavioral symptom of ASD.

Therefore, the present invention provides for the prevention of gastrointestinal hyperpermeability or the reduction of gastrointestinal hyperpermeability, and thereby the treatment or amelioration of one or more symptoms of a disorder associated with gastrointestinal hyperpermeability, for example ASD, Crohn's disease, celiac disease, ulcerative colitis, diabetes type 1, arthritis including spondylarthrites, psoriatic arthritis, and/or inflammation, in particular gastrointestinal inflammation.

Suitably, the subject has, or is susceptible to or at risk of developing, one or more symptoms of ASD, such as a behavioural symptom and/or a gastrointestinal disorder.

Suitably, the subject has been diagnosed with ASD or a symptom thereof. Suitably, a method of the first aspect comprises a step of diagnosing a subject with ASD or a symptom thereof.

Suitably, the subject is susceptible to, or has been diagnosed with, a behavioural symptom of ASD, and the cannabinoid is provided for use in a method of preventing or treating said behavioral symptom of ASD.

Suitably, the subject is susceptible to, or has been diagnosed with, a gastrointestinal symptom of ASD, and the cannabinoid is provided for use in a method of preventing or treating said gastrointestinal symptom of ASD.

Suitably, the subject has been diagnosed with gastrointestinal hyperpermeability or risk of developing gastrointestinal hyperpermeability. The subject may have one or more symptoms of gastrointestinal hyperpermeability. The subject may be suspected of having gastrointestinal hyperpermeability.

Suitably, the subject is susceptible to, or has been diagnosed with a combination of a behavioural symptom and a gastrointestinal disorder (symptom), and the cannabinoid is provided for use in a method of preventing or treating said combination of behavioral and gastrointestinal symptoms.

Suitably, the subject is susceptible to, or has been diagnosed with crohn's disease, celiac disease, ulcerative colitis, diabetes type 1, arthritis including spondylarthrites and psoriatic arthritis. Suitably, the subject is susceptible to, or has been diagnosed with crohn's disease, celiac disease, ulcerative colitis, diabetes type 1, arthritis including spondylarthrites, psoriatic arthritis and/or inflammation, in particular gastrointestinal inflammation, and the cannabinoid is provided for use in a method of preventing or treating Crohn's disease, celiac disease, ulcerative colitis, diabetes type 1, or arthritis including spondylarthrites, psoriatic arthritis, and/or inflammation, in particular gastrointestinal inflammation.

The cannabinoid may be CBD. The cannabinoid may be THC. The cannabinoid may comprise a combination of CBD and THC. The cannabinoid may consist of a combination of CBD and THC. Suitably, a combination of CBD and THC may be used to prevent or treat gastrointestinal hyperpermeability in a subject, suitably in a subject who has been diagnosed with ASD or a symptom thereof, or is susceptible to ASD or a symptom thereof. The cannabinoid may be provided as a composition or formulation, as described herein. Suitably, the composition is for oral administration.

Most suitably, the first aspect of the invention provides a combination of CBD and THC for use in the prevention or treatment of gastrointestinal hyperpermeability in a subject who has been diagnosed with, or is susceptible to, a behavioral symptom of ASD. A behavioral symptom may be irritability, aggressive response, hyperactivity, non-typical social behaviour, non-typical exploratory behaviour and/or non-typical stereotypic behaviour.

A suitable human dosage of CBD may be about 0.1 to 20 mg/kg, suitably 0.5 to 15 mg/kg, suitably 0.8 to 12 mg/kg, suitably 1 to 10 mg/kg, or any range using the aforementioned upper or lower limits, or any integer which falls in any of the aforementioned ranges. Most suitably, a suitable dose may be 1.6, 4.8 & 9.6 mg/kg of body weight of CBD. A suitable human dose of THC may be 0.05 to 6 mg/kg, suitably 0.1 to 5 mg/kg, most suitably 0.1 to 4 mg/kg body weight. A suitable human dosage may be about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5 or 6 mg/kg, any range formed from any of the aforementioned integers as lower and upper limits, or any integer which falls in any of the aforementioned ranges.

In a second aspect of the invention, there is provided a cannabinoid for use in a method of preventing or treating a symptom of an ASD in a subject, wherein the subject has been diagnosed as having gastrointestinal hyperpermeability or is at risk or susceptible to gastrointestinal hyperpermeability.

Also provided is a method of preventing or treating a symptom of ASD in a subject, wherein the method comprises administering a cannabinoid to the subject, wherein the subject has, or has been tested as having, gastrointestinal hyperpermeability.

Suitably, the cannabinoid may prevent or reduce gastrointestinal hyperpermeability by reducing or preventing colonic muscle thinning, increasing colonic muscle thickness, and/or maintaining colonic muscle thickness.

Suitably, the subject has been diagnosed with ASD or a symptom thereof. Suitably, a method of the second aspect comprises a step of diagnosing a subject with ASD or a symptom thereof. Suitably, the symptom of ASD is a behavioural symptom. Suitably, the symptom of ASD is a gastrointestinal disorder. Suitably, the symptom of ASD is a combination of a behavioural symptom and a gastrointestinal disorder (symptom).

Suitably, the subject has, or is susceptible to or at risk of developing, one or more symptoms of ASD, such as a behavioural symptom and/or a gastrointestinal disorder. Suitably, the subject is susceptible to, or has been diagnosed with, a behavioural symptom of ASD, and the cannabinoid is provided for use in a method of preventing or treating said behavioral symptom of ASD.

Suitably, the subject is susceptible to, or has been diagnosed with, a gastrointestinal symptom of ASD, and the cannabinoid is provided for use in a method of preventing or treating said gastrointestinal symptom of ASD.

Suitably, the subject is susceptible to, or has been diagnosed with a combination of a behavioural symptom and a gastrointestinal disorder (symptom), and the cannabinoid is provided for use in a method of preventing or treating said combination of behavioral and gastrointestinal symptoms.

Suitably the subject has one or more symptoms of gastrointestinal hyperpermeability. Suitably the subject has been diagnosed as having gastrointestinal hyperpermeability or diagnosed as being at risk or susceptible to gastrointestinal hyperpermeability. The subject may have one or more symptoms of gastrointestinal hyperpermeability. The subject may be suspected of having gastrointestinal hyperpermeability.

The cannabinoid may be CBD. The cannabinoid may be THC. The cannabinoid may comprise a combination of CBD and THC. The cannabinoid may consist of a combination of CBD and THC. Suitably, a combination of CBD and THC may be used to prevent or treat gastrointestinal hyperpermeability in a subject, suitably a subject has been diagnosed with ASD or a symptom thereof, or susceptible to ASD or a symptom thereof. The cannabinoid may be provided as a composition or formulation, as described herein. Suitably, the composition is for oral administration.

A suitable human dosage may be about 0.1 to 20 mg/kg, suitably 0.5 to 15 mg/kg, suitably 0.8 to 12 mg/kg, suitably 1 to 10 mg/kg, or suitably may be about 0.1, 0.2, 0.3, 0.4, 0.5, 0.6, 0.7, 0.8, 0.9, 1, 1.5, 2, 2.5, 3, 3.5, 4, 4.5, 6, 6.5, 7, 7.5, 8, 8.5, 9, 9.5, 10, 10.5, 11, 12, 13, 14, 15, 16, 17, 18, 19 or 20 mg/kg, any range formed from any of the aforementioned integers as lower and upper limits, or any integer which falls in any of the aforementioned ranges. Most suitably, a suitable dose may be 1.6, 4.8 & 9.6 mg/kg of body weight of CBD. A suitable human dose of THC for a human subject may be 0.05 to 6 mg/kg, suitably 0.1 to 5 mg/kg, most suitably 0.1 to 4 mg/kg body weight. A suitable human dosage may be about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5 or 6 mg/kg, any range formed from any of the aforementioned integers as lower and upper limits, or any integer which falls in any of the aforementioned ranges.

Most suitably, the second aspect provides a combination of CBD and THC for use in the prevention or treatment of a symptom of ASD in a subject who has been diagnosed with, or is susceptible to, a gastrointestinal hyperpermeability. The symptom of ASD may be behavioral symptom, for example irritability, aggressive response, hyperactivity, non-typical social behaviour, non-typical exploratory behaviour and/or non-typical stereotypic behaviour. The combination may be provided as a composition or formulation, as described herein. Suitably, the combination is an oral composition.

Also provided, in a third aspect, is the use of a cannabinoid in the prevention or reduction in gastrointestinal inflammation in a subject.

Also provided is a method of preventing or treating gastrointestinal inflammation in a subject, wherein the method comprises administering a cannabinoid to the subject.

The cannabinoid may prevent or reduce gastrointestinal hyperpermeability.

Suitably, the cannabinoid may prevent or reduce gastrointestinal hyperpermeability by reducing or preventing colonic muscle thinning, increasing colonic muscle thickness, and/or maintaining colonic muscle thickness.

The subject may have, or may have been tested as having gastrointestinal hyperpermeability. Suitably, the subject has, or is susceptible to, or at risk of developing, gastrointestinal hyperpermeability. The subject may have one or more symptoms of gastrointestinal hyperpermeability. The subject may be suspected of having gastrointestinal hyperpermeability.

Suitably, the subject may have, or may have been tested as having, gastrointestinal inflammation. Suitably, the subject has been diagnosed with, or is susceptible to, or at risk of developing, gastrointestinal inflammation.

Suitably, the subject has been diagnosed with ASD or a symptom thereof. Suitably, a method of the third aspect comprises a step of diagnosing a subject with ASD or a symptom thereof. Suitably, the symptom of ASD is a behavioural symptom. Suitably, the symptom of ASD is a gastrointestinal disorder. Suitably, the symptom of ASD is a combination of a behavioural symptom and a gastrointestinal disorder (symptom).

The subject may have, may be susceptible to, or may have been diagnosed with one or more of Crohn's disease, celiac disease, ulcerative colitis, diabetes type 1, arthritis including spondyloarthritis and psoriatic arthritis, or inflammation. Suitably, a method of the first aspect comprises a step of diagnosing a subject with one or more of Crohn's disease, celiac disease, ulcerative colitis, diabetes type 1, arthritis including spondyloarthritis and psoriatic arthritis, or inflammation.

Suitably, the third aspect for the invention is suitable for the prevention or treatment of ASD or a symptom thereof, Crohn's disease, celiac disease, ulcerative colitis, diabetes type 1, arthritis including spondylarthrites, psoriatic arthritis and/or inflammation, or one or more symptoms of an aforementioned condition.

The cannabinoid may be CBD. The cannabinoid may be THC. The cannabinoid may comprise a combination of CBD and THC. The cannabinoid may consist of a combination of CBD and THC. Suitably, a combination of CBD and THC may be used to prevent or treat gastrointestinal inflammation in a subject. The cannabinoid may be provided as a composition or formulation, as described herein. Suitably, the composition is for oral administration.

Most suitably, the second aspect provides a combination of CBD and THC for use in the prevention or treatment of gastrointestinal inflammation in a subject, wherein the subject has been diagnosed with ASD or a symptom thereof, Crohn's disease, celiac disease, ulcerative colitis, diabetes type 1, arthritis including spondylarthrites, psoriatic arthritis and/or inflammation, or one or more symptoms of an aforementioned condition.

The symptom of ASD may be behavioral symptom, for example irritability, aggressive response, hyperactivity, non-typical social behaviour, non-typical exploratory behaviour and/or non-typical stereotypic behaviour.

Most suitably, the third aspect of the invention provides a combination of CBD and THC for use in the prevention or treatment of a gastrointestincal inflammation in a subject, wherein the subject is susceptible to, or has been diagnosed with ASD or a symptom thereof. The combination may be provided as a composition or formulation, as described herein. Suitably, the combination is an oral composition.

In accordance with a fourth aspect of the present invention, there is provided a cannabinoid for use in the prevention or treatment of a symptom of ASD in a subject, by preventing or reducing gastrointestinal hyperpermeability.

Also provided is a method of preventing or treating a symptom of ASD in a subject, wherein the method comprises administering a cannabinoid to the subject, wherein the cannabinoid prevents or reduces gastrointestinal hyperpermeability.

Suitably, the cannabinoid may prevent or reduce gastrointestinal hyperpermeability by reducing or preventing colonic muscle thinning, increasing colonic muscle thickness, and/or maintaining colonic muscle thickness.

Suitably, the subject has been diagnosed with ASD or a symptom thereof. Suitably, a method of the fourth aspect comprises a step of diagnosing a subject with ASD or a symptom thereof. Suitably, the subject may be suspected of having ASD or a symptom thereof.

Suitably, the symptom of ASD is a behavioural symptom. Suitably, the symptom of ASD is a gastrointestinal disorder. Suitably, the symptom of ASD is a combination of a behavioural symptom and a a gastrointestinal disorder (symptom).

The subject may have, or may have been tested as having gastrointestinal hyperpermeability. Suitably, the subject has, or is susceptible to, or at risk of developing, gastrointestinal hyperpermeability. The subject may have one or more symptoms of gastrointestinal hyperpermeability. The subject may be suspected of having gastrointestinal hyperpermeability.

Suitably, in the first aspect of the invention, the cannabinoid is CBD, THC, or a combination of CBD and THC. The cannabinoid may consist of a combination of CBD and THC. The cannabinoid may be provided as a composition or formulation, as described herein. Suitably, the composition is for oral administration.

Therefore there is provided a cannabinoid for use in the prevention or treatment of a symptom of ASD in a subject, by preventing or reducing colonic muscle thinning, reducing or preventing colonic mucosal thinning, increasing colonic muscle thickness, increasing colonic mucosal thickness, and/or maintaining colonic muscle or mucosal thickness.

Most suitably, the fourth aspect of the invention provides a combination of CBD and THC for use in the prevention or treatment of a behavioral symptom of ASD in a subject, by preventing or reducing gastrointestinal hyperpermeability. A behavioral symptom may be irritability, aggressive response, hyperactivity, non-typical social behaviour, non-typical exploratory behaviour and/or non-typical stereotypic behaviour. The combination may be provided as a composition or formulation, as described herein. Suitably, the combination is an oral composition.

A suitable human dosage of CBD may be about 0.1 to 20 mg/kg, suitably 0.5 to 15 mg/kg, suitably 0.8 to 12 mg/kg, suitably 1 to 10 mg/kg, or any range using the aforementioned upper or lower limits, or any integer which falls in any of the aforementioned ranges. Most suitably, a suitable dose may be 1.6, 4.8 & 9.6 mg/kg of body weight of CBD. A suitable human dose of THC may be 0.05 to 6 mg/kg, suitably 0.1 to 5 mg/kg, most suitably 0.1 to 4 mg/kg body weight. A suitable human dosage may be about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5 or 6 mg/kg, any range formed from any of the aforementioned integers as lower and upper limits, or any integer which falls in any of the aforementioned ranges.

In accordance with a fifth aspect of the present invention, there is provided a cannabinoid for use in the prevention or reduction in gastrointestinal inflammation in a subject, by preventing or reducing gastrointestinal hyperpermeability.

Also provided is a method of preventing or treating gastrointestinal inflammation in a subject, wherein the method comprises administering a cannabinoid to the subject, wherein the cannabinoid prevents or reduces gastrointestinal hyperpermeability.

Suitably, the subject has been diagnosed with ASD or a symptom thereof. Suitably, a method of the first aspect comprises a step of diagnosing a subject with ASD or a symptom thereof. Suitably, the subject may be suspected of having ASD or a symptom thereof.

Suitably, the symptom of ASD is a behavioural symptom. Suitably, the symptom of ASD is a gastrointestinal disorder. Suitably, the symptom of ASD is a combination of a a behavioural symptom and a a gastrointestinal disorder (symptom).

The subject may have, may be susceptible to, or may have been diagnosed with one or more of Crohn's disease, celiac disease, ulcerative colitis, diabetes type 1, arthritis including spondyloarthritis and psoriatic arthritis, or inflammation, in particular gastrointestinal inflammation. Suitably, a method of the first aspect comprises a step of diagnosing a subject with one or more of Crohn's disease, celiac disease, ulcerative colitis, diabetes type 1, arthritis including spondyloarthritis and psoriatic arthritis, or inflammation, in particular gastrointestinal inflammation. Suitably, the fifth aspect for the invention is suitable for the prevention or treatment of ASD, Crohn's disease, celiac disease, ulcerative colitis, diabetes type 1, arthritis including spondylarthrites and psoriatic arthritis.

The subject may have, or may have been tested as having gastrointestinal hyperpermeability. Suitably, the subject has, or is susceptible to, or at risk of developing, gastrointestinal hyperpermeability. The subject may have one or more symptoms of gastrointestinal hyperpermeability. The subject may be suspected of having gastrointestinal hyperpermeability.

Suitably, in the fifth aspect of the invention, the cannabinoid is CBD, THC, or a combination of CBD and THC. The cannabinoid may consist of a combination of CBD and THC. The cannabinoid may be provided as a composition or formulation, as described herein. Suitably, the composition is for oral administration.

Most suitably, the fifth aspect of the invention provides a combination of CBD and THC for use in the prevention or treatment of a gastrointestinal inflammation in a subject, by preventing or reducing gastrointestinal hyperpermeability. Suitably, the subject is susceptible to, or has been diagnosed with ASD or a symptom thereof. The combination may be provided as a composition or formulation, as described herein. Suitably, the combination is an oral composition.

In accordance with a sixth aspect of the present invention, there is provided a cannabinoid for use in the prevention or treatment of a symptom of ASD in a subject, by preventing or reducing colonic muscle thinning.

Also provided is a method of preventing or treating a symptom of ASD in a subject, wherein the method comprises administering a cannabinoid to the subject, wherein the cannabinoid prevents or reduces colonic muscle thinning.

In accordance with a seventh aspect of the present invention, there is provided a cannabinoid for use in the prevention or treatment of a symptom of ASD in a subject, wherein the subject has, or has been tested as having, is susceptible to, or is at risk of developing, colonic muscle thinning.

Also provided is a method of preventing or treating a symptom of ASD in a subject, wherein the method comprises administering a cannabinoid to the subject, wherein the subject has, or has been tested as having, gastrointestinal hyperpermeability.

Therefore there is provided a cannabinoid for use in the prevention or treatment of a symptom of ASD in a subject, by preventing or reducing colonic muscle thinning, increasing colonic muscle thickness, and/or maintaining colonic muscle thickness.

Suitably, the cannabinoid may prevent or reduce colonic muscle thinning or increase colonic muscle thickness, and/or maintain colonic muscle thickness. The effect of preventing or reducing, or treating colonic muscle thinning may be useful in the prevention or treatment of gastrointestinal hyperpermeability, and conditions including ASD or symptoms thereof, in particular behavioural symptoms.

Suitably, the sixth and seventh aspects of the invention may be useful in the prevention or treatment of a gastrointestinal symptom or disorder, ASD or a symptom thereof such as a gastrointestinal symptom of ASD, and/or a behavioral symptom of ASD. A gastrointestinal symptom or disorder may include Crohn's disease, celiac disease, ulcerative colitis, diabetes type 1, arthritis including spondyloarthritis and psoriatic arthritis, and/or inflammation, in particular gastrointestinal inflammation.

In the sixth or seventh aspects, a subject may have been diagnosed with ASD or a symptom thereof. The subject may have been diagnosed with one or more of Crohn's disease, celiac disease, ulcerative colitis, diabetes type 1, arthritis including spondyloarthritis and psoriatic arthritis, or inflammation, in particular gastrointestinal inflammation. Suitably, the sixth or seventh aspect may comprises a step of diagnosing a subject with one or more of ASD or a symptom thereof, Crohn's disease, celiac disease, ulcerative colitis, diabetes type 1, arthritis including spondyloarthritis and psoriatic arthritis, or inflammation, in particular gastrointestinal inflammation.

The subject may have, or may have been tested as having, gastrointestinal hyperpermeability. Suitably, the subject has been diagnosed with, or is susceptible to, or at risk of developing, gastrointestinal hyperpermeability. The subject may have one or more symptoms of gastrointestinal hyperpermeability. The subject may be suspected of having gastrointestinal hyperpermeability.

Suitably, the subject may have, or may have been tested as having, gastrointestinal inflammation. Suitably, the subject has been diagnosed with, or is susceptible to, gastrointestinal inflammation. The subject may have one or more symptoms of gastrointestinal inflammation. The subject may be suspected of having gastrointestinal inflammation.

Suitably, in the first aspect of the invention, the cannabinoid is CBD, tetrahydrocannabinol (THC), or a combination of CBD and THC. The cannabinoid may consist of a combination of CBD and THC. The cannabinoid may be provided as a composition or formulation, as described herein. Suitably, the composition is for oral administration.

In an eighth aspect, there are provided are formulations and compositions comprising a cannabinoid, at an effective dose for use in a method of the invention, for example treating or preventing gastrointestinal hyperpermeability or a symptom of ASD as described herein.

Also provided are kits comprising a cannabinoid, for use in a method of the invention as described herein.

### Autism Spectrum Disorder

By ASD herein is meant a neurological and developmental disorder, also referred to as autism and may include Asperger's syndrome, childhood disintergrative disorder, and pervasive developmental disorder, and social communication disorder. A subject with ASD may have persistent difficulties with social interaction and/or communication, and restricted or repetitive patterns of behaviours, interests or activities.

Herein, where a subject is referred to as having ASD or having been diagnosed with ASD, the subject may have one or more symptoms of ASD.

A symptom of ASD may be a behavioural symptom or may be a gastrointestinal disorder symptom. Suitably, ASD is diagnosed by the presence of one or more behavioral symptoms, which may be present in combination with one or more gastrointestinal symptoms. A behavioural symptom may be, but is not limited to, a symptom selected from restlessness, aggression, rage, agitation, self-injury, tantrums, stress, anxiety, irritability, communication problems, defiance, disruptive behaviours, psychosis, lethargy, seizures, hypersensitivity, inattentiveness, stereotyped or repetitive behaviours, speech problems, sleep problems, social deficits, insomnia, learning difficulties, daily functioning difficulties, cognitive impairment or delay, attention disorder, depression, lack of concentration, dyslexia, epilepsy, motor tics, and/or vocal tics. Gastrointestinal disorder symptoms may be selected from abdominal pain or discomfort, bloating, constipation, flatulence, lack of appetite/weight loss, incontinence, fussy eating, or diarrhoea. Diagnosis or testing for ASD may be based on observations of behaviour and assessment of the presence or absence of one or more behavioral symptoms, for example as described herein or other symptoms not listed herein which are indicative of ASD. Diagnostic criteria are set out in diagnostic manuals ICD-10 and DSM-5 of the National Autistic Society (UK). Diagnostic tools include, by way of example and without limitation, the Diagnostic Interview for Social and Communication Disorders (DISCO), Autism Diagnostic Interview (ADI), the Autism Diagnostic Observation Schedule (ADOS) and Developmental, Dimensional and Diagnostic Interview (3Di). Diagnosis may also be based on factors such as a sibling or relative of the subject having ASD or symptoms thereof, genetic or chromosomal abnormalities for example fragile X syndrome or tuberous sclerosis, complications at birth, and/or or geriatric parents. Other suitable diagnostic methods may be known or available to a person skilled in the art, and may be used in relation to the presented invention.

Diagnosis may be used to provide a severity level for ASD. Severity may be categorised as Level 1, Level 2 or Level 3 based on defined characteristics of social communication behaviours and restricted, repetitive behaviours.

| Severity | | Restricted, |
|---|---|---|
| level | Social communication | repetitive behaviours |
| Level 3 | Severe deficits in verbal | Inflexibility of behaviour, |
| “Requiring | and nonverbal social | extreme difficulty coping |
| very | communication skills | with change, or other |
| substantial | cause severe impairments | restricted/repetitive |
| support” | in functioning, very | behaviours markedly interfere |
| | limited initiation of social | with functioning in all |
| | interactions, and minimal | spheres. Great distress/ |
| | response to social overtures | difficulty changing focus or |
| | from others. For example, | action. |
| | a person with few words of | |
| | intelligible speech who | |
| | rarely initiates interaction | |
| | and, when he or she does, | |
| | makes unusual approaches | |
| | to meet needs only and | |
| | responds to only very | |
| | direct social approaches | |
| Level 2 | Marked deficits in verbal | Inflexibility of behaviour, |
| “Requiring | and nonverbal social | difficulty coping with |
| substantial | communication skills; | change, or other restricted/ |
| support” | social impairments | repetitive behaviours appear |
| | apparent even with | frequently enough to be |
| | supports in place; limited | obvious to the casual |
| | initiation of social | observer and interfere with |
| | interactions; and reduced | functioning in a variety of |
| | or abnormal responses to | contexts. Distress and/or |
| | social overtures from | difficulty changing focus or |
| | others. For example, a | action. |
| | person who speaks simple | |
| | sentences, whose | |
| | interaction is limited to | |
| | narrow special interests, | |
| | and how has markedly odd | |
| | nonverbal communication. | |
| Level 1 | Without supports in place, | Inflexibility of behavior |
| “Requiring | deficits in social | causes significant interference |
| support” | communication cause | with functioning in one or |
| | noticeable impairments. | more contexts. Difficulty |
| | Difficulty initiating social | switching between activities. |
| | interactions, and clear | Problems of organization and |
| | examples of atypical or | planning hamper |
| | unsuccessful response to | independence. |
| | social overtures of others. | |
| | May appear to have | |
| | decreased interest in social | |
| | interactions. For example, | |
| | a person who is able to | |
| | speak in full sentences and | |
| | engages in communication | |
| | but whose to- and-fro | |
| | conversation with others | |
| | fails, and whose attempts | |
| | to make friends are odd | |
| | and typically unsuccessful. | |

### Gastrointestinal Hyperpermeability

Increased gastrointestinal permeability may also be referred to herein as gut hyperpermeability, high intestinal permeability, or leaky gut.

Increased gastrointestinal permeability is a condition in which the intestinal lining becomes more permeable than normal, typically through damage, such that particles which are normally unable to cross the epithelial barrier of the gut are able to escape into the circulatory system. Such particles may include for example, larger, undigested food particles, proteins, and intestinal microbes. Damage may include thinning of the intestinal (e.g. colonic or small intestine) muscle wall.

Herein, where a subject is referred to as having gastrointestinal hyperpermeability, it may mean that the subject displays one or more symptoms of gastrointestinal hyperpermeability. The subject may have been diagnosed with gastrointestinal hyperpermeability. A method of diagnosing may include one or more of the following tests, or any other test available in the art.

Gastrointestinal hyperpermeability may be tested for, or diagnosed, using any suitable test or combination of tests. A test will be available to a person skilled in the art, and are not limited to those described herein. Each test will have available parameters for determining whether gut permeability is outside of a healthy, non-leaky range. Examples of tests for assessing gastrointestinal hyperpermeability in a subject include using non-metabolizable probes which pass across the mucosal barrier and are excreted into the urine after being absorbed into the circulatory system. Quantitation of the probe in a timed urine collection provides a measure of the amount of absorbed probe, which in turn provides an indication of abnormality in intestinal permeability. Suitable probes include, but are not limited to, sucrose, lactulose, PEG-400, sucralose, Cr-EDTA, and FITC dextran. A probe may be selected depending on its site of absorption (small intestine, gastro-duodenum, colon or whole gut). A test may use a combination of probes, for example a sugar and non-sugar probe or a monosaccharide and a disaccharide. Combinations include for example lactulose/mannitol, lactulose/L-mannitol and cellobiose/mannitol (small intestine) and Cr-EDTA/mannitol, sucralose/mannitol and sucralose/L-rhannose (whole gut). Intestinal permeability is measured as a ratio of excretion of the two probes into the urine. The concentration of one or more probes present in the urine can be measured using enzyme assay, high performance liquid chromatography (HPLC), ion-exchange chromatography in combination with mass spectrometry and liquid chromatography with mass spectrometry. If PEG has been used as a probe, then the concentration of PEG can be assessed using capillary column gas chromatography. Suitably, a combination of lactulose and mannitol is used. Suitably, measurement is made using HPLC. The excretion of probes may be expressed as % excretion of ingested dose for each probe. Where the probes are lactulose and mannitol, a ratio of excretion of lactulose to excretion of mannitol for a healthy subject may be in the range of 0.002 to 0.25. For example if 2 units of lactulose were excreted and 8 of mannitol, then a ratio of 0.25 would be derived. As permeability increases, more lactulose is excreted and the ratio trends toward unity (i.e. 1:1).

Other tests include a blood test, which comprises analysing a blood sample for presence or absence of gut microbiota, for example gut bacteria, or for bacterial products such as endotoxins. Any suitable gut biomarker may be tested for, including a specific protein, antibody and/or endotoxin. The protein zonulin is an example of a suitable biomarker for determining gut permeability. A blood test may include quantitation of gut microbiota or biomarkers as a measure of the extent of gut permeability. A test may comprise a tissue biopsy, wherein a sample of intestinal tissue may be removed and examined in an Ussing chamber. An electrical current is used to measure ion transport across the intestinal barrier. The ion transport measurement is parallel to water transport, and the amount of ion transport provides a measure of the permeability of the tissue. Confocal endomicroscopy may be used to observe the intestinal lining in high resolution and magnification. For example, a loss in intestinal muscle thickness may be indicative of gastrointestinal hyperpermeability. A contrast fluid may be administered, to highlight and gaps in the gut lining.

Suitably, a test for intestinal permeability is conducted on a subject who may be required to fast overnight or for a suitable period of time.

Suitably a subject who has been diagnosed as having gastointrestinal hyperpermeabilioty has at least one test marker which is outside of a healthy range.

### Colonic Muscle Thickness

Colonic muscle thinning is a condition in which the thickness of the colon muscle wall becomes reduced. This may lead to the intestinal lining becomes more permeable than normal, typically through damage, such that particles which are normally unable to cross the epithelial barrier of the gut are able to escape into the circulatory system. Such particles may include for example, larger, undigested food particles, proteins, and intestinal microbes. Damage may include thinning of the intestinal (e.g. colonic or small intestine) muscle wall.

Herein, where a subject is referred to as having colonic muscle thinning, it may mean that the subject displays one or more symptoms of impaired gastrointestinal function. One or more symptoms may be the same as the symptoms of gastrointestinal hyperpermeability. The subject may have been diagnosed with colonic muscle thinning. A method of diagnosing may include one or more of the following tests, or any other test available in the art.

Colonic muscle thinning may be tested for, or diagnosed, using any suitable test or combination of tests. A test will be available to a person skilled in the art, and are not limited to those described herein. Each test will have available parameters for determining whether colonic wall thickness is outside of a healthy range. The tests may be the same as the tests for gastrointestinal hyperpermeability.

Confocal endomicroscopy may be used to observe the colon wall in high resolution and magnification. For example, a loss in colon muscle thickness may be indicative of gastrointestinal hyperpermeability. A contrast fluid may be administered, to highlight and gaps in the gut lining.

Suitably, a test for colon wall thickness is conducted on a subject who may be required to fast overnight or for a suitable period of time.

Suitably a subject who has been diagnosed as having impaired colon muscle thickness has at least one test marker which is outside of a healthy range.

### Cannabinoids

A cannabinoid as referred to herein is a compound which is capable of modification of the endocannabinoid system in a subject. Thus, a cannabinoid may be any compound which is an agonist or antagonist or modulator of a receptor in the endocannabinoid system (for example CB, CB2 or GPR55) or is capable of affecting endogenous endocannabinoids synthesis or degredation. A cannabinoid may be a phytocannabinoid. A phytocannabinoid may be derived from the Cannabis plant, and may be for example cannabidiol (CBD), cannabinol (CBN), cannabigerol (CBG), cannabicyclol (CBL), cannabinigerovarin (CBDV), cannabidiolic acid (CBDA) and cannabichromene (CBC), tetrahydrocannabinol (THC), or tetrahydrocannabivarin (THCV).

Alternatively, a phytocannabinoid may be derived from another plant for example Echinacea purpurea, Echinacea angustifolia, Acmella oleracea, Helichrysum umbraculigerum, and/or Radula marginata. A cannabinoid which is not derived from a Cannabis plant may be a lipophilic alkamides (alkylamide) from an Echinacea species. Suitably, a non-Cannabis cannabinoid may be the cis/trans isomers dodeca-2E,4E,8Z, 10E/Z-tetraenoic-acid-isobutylamide. Alternatively, a cannabinoid may be an endocannabinoid, which naturally occurs in a human or animal body. Endocannabinoids include for example 2-Arachidonoylglycerol (2-AG), 2-Arachidonyl glyceryl ether (noladin ether), N-Arachidonoyl dopamine (NADA), Virodhamine (OAE), Arachidonoylethanolamine (Anandamide or AEA), and Lysophosphatidylinositol (LPI).

Alternatively, a cannabinoid may be synthetic. A synthetic cannabinoid may be a synthetic endocannabinoid or a synthetic phytocannabinoid. Examples of synthetic cannabinoids include, without limitation, JWH-073, CP-55940, Dimethylheptylpyran, HU-210, HU-211, HU-331, SR144528, WIN 55,212-2, JWH-133, Levonantradol (Nantrodolum), or AM-2201 a potent cannabinoid receptor agonist, or THC (Naramco). Other suitable synthetic cannabinoids may be developed and may be available to a person skilled in the art, and are included within the scope of the present invention. Lists of suitable synthetic cannabinoids may be available, for example Cayman Chemicals (MI, USA) Product Information; Synthetic Cannabinoid Screening Library, Item No. 9002891; Batch No, 0641180 https://cdn.caymanchem.com/cdn/insert/9002891.pdf).

A cannabinoid may be provided in an acid form or a non-acid (decarboxylated) form. An acid form of a cannabinoid may be referred to by adding the letter “a” to the cannabinoid acronym, for example THCa, or CBDa. A cannabinoid for use in the present invention may be in decarboxylated form, or may be a combination of acid and non-acid forms. A cannabinoid for use in the present invention may comprise at least or no more than 5, 10, 20, 30, 40, 50, 60, 70, 80, or 90% of the acid form. Alternatively, a cannabinoid for use in the present invention may comprise at least, or nor more than 5, 10, 20, 30, 40, 50, 60, 70, 80, or 90% of the non-acid form.

A primary cannabinoid is a cannabinoid which is primarily responsible for the therapeutic effect. In the context of the present invention, a primary cannabinoid may be CBD. When present, THC may also be referred to as being a primary cannabinoid. A formulation, composition or kit of the present invention may comprise other cannabinoids in addition to one or more primary cannabinoids. Other cannabinoids which are present may be referred to as a non-primary cannabinoid, meaning that they are not responsible for the therapeutic effect, as defined herein.

A cannabinoid for use in the present invention may be obtained from any suitable source. It may be extracted from a plant or it may be synthesized. A cannabinoid for use in the present invention may be entirely plant derived, entirely synthetic, or may be a blend of plant-derived and synthetic, in any suitable proportion. Methods are available in the art for extraction of cannabinoids from a plant, and synthesis of a cannabinoid. Purification of a cannabinoid may be performed using any suitable method, for example an ethanol or carbon dioxide based extraction. In an embodiment, a combination of a plant derived cannabinoid and a synthetic cannabinoid may be used. In an embodiment of the aspects and embodiments described herein, a plant derived CBD and a synthetic THC may be used.

A cannabinoid for use in the invention may be substantially pure, or may comprise impurities, for example plant material. Suitably, a cannabinoid for use in the present invention comprises about, or at least, 50%, 60%, 70%, 80%, 90% or 95% cannabinoid, suitably a primary cannabinoid. Suitably, a cannabinoid for use in the present invention comprises a CBD. Suitably, a cannabinoid for use in the present invention comprises about or at least 50%, 60%, 70%, 80%, 90% or 95% of a CBD. Suitably, a cannabinoid for use in the present invention comprises a CBN. Suitably, a cannabinoid for use in the present invention comprises about, or at least, 50%, 60%, 70%, 80%, 90% or 95% of a CBN. Suitably, a cannabinoid for use in the present invention comprises a CBG. Suitably, a cannabinoid for use in the present invention comprises about, or at least, 50%, 60%, 70%, 80%, 90% or 95% of a CBG. Suitably, a cannabinoid for use in the present invention comprises a CBL. Suitably, a cannabinoid for use in the present invention comprises about or at least 50%, 60%, 70%, 80%, 90% or 95% of a CBL. Suitably, a cannabinoid for use in the present invention comprises a CBC. Suitably, a cannabinoid for use in the present invention comprises about or at least 50%, 60%, 70%, 80%, 90% or 95% of a CBC. Suitably, a cannabinoid for use in the present invention comprises a CBDV. Suitably, a cannabinoid for use in the present invention comprises about or at least 50%, 60%, 70%, 80%, 90% or 95% of a CBDV. Suitably, a cannabinoid for use in the present invention comprises a THC. Suitably, a cannabinoid for use in the present invention comprises about or at least 50%, 60%, 70%, 80%, 90% or 95% of a THC. Suitably, a cannabinoid for use in the present invention comprises a THCV. Suitably, a cannabinoid for use in the present invention comprises at least 50%, 60%, 70%, 80%, 90% or 95% of a THCV.

A cannabinoid for use in the present invention may be a single cannabinoid or may be a combination of two or more different cannabinoids. For example, a cannabinoid for use in the present invention may comprise any combination of two or more phytocannabinoids, endocannabinoids or synthetic cannabinoids. Where a combination of two or more primary cannabinoids are provided for use in the present invention, a cannabinoid may comprise about or at least 50%, 60%, 70%, 80%, 90% or 95% of the combination of the two or more primary cannabinoids. For example, a cannabinoid may comprise about, or at least 50%, 60%, 70%, 80%, 90% or 95% of a combination of CBD and THC.

### Compositions

A cannabinoid may be provided as a composition, for example a nutritional or pharmaceutical composition. A composition comprising a cannabinoid may comprise the cannabinoid in a concentration of at least, or no more than about 0.5%, 1%, 2%, 3%, 4%, 5%, 10%, 20%, 30%, 40%, 50%, 60|%, 70%, 80% or 90% by weight. Suitably, a cannabinoid may be present at at least, or no more than, or about 40, 45, 50, 55 or 60% by weight of the composition, most suitably 40, 45 or 50% by weight of the composition where the composition is a granular composition.

A composition comprising a cannabinoid may comprise water. A composition may comprise at least, or no more than, 1%, 5%, 10%, 20%, 30%, 40% or 50% of water by weight. A composition comprising water may be an emulsion, such as a nano or micro emulsion. A composition comprising water may additionally comprise an agent to solubilise the cannabinoid and/or THC, for example PEG or TPGS, or any suitable agent.

A composition may comprise a further therapeutic agent. A further therapeutic agent may be a different cannabinoid, or may be a different agent which targets the endocannabinoid system or a different physiological system, for example a neurological system or a gastrointestinal system. A further therapeutic agent may be THC. A cannabinoid may comprise CBD, CBN, CBG, CBL, CBDV or CBC, and may be administered in combination with THC. The CBD, CBN, CBG, CBL or CBC may be provided separately to the THC, or may be provided as a combined formulation. Alternatively, the therapeutic (or active) agents in a composition may consist of CBD and THC.

A composition comprising a cannabinoid may comprise an additive. An additive may be selected from, but is not limited to, a preservative, antioxidant, diluent, emulsifier, texturizer, excipient, carrier or surfactant. A suitable additive may be an edible additive, suitably a food approved additive. For example, an edible additive may be plant material (such as cannabis plant material), a plant oil, plant extract, or honey. A suitable additive may be a pharmaceutically approved additive such as a pharmaceutical excipient.

A surfactant may be selected from a phospholipid, glycolipid, glyceride, or a combination thereof.

An antioxidant may be selected from, but is not limited to, citric acid, ascorbic acid, tartaric acid, phosphoric acid, sodium metabisulfite, thiol derivatives, buylated hydroxy anisole, butylated hydroxy toluene, tertiary butyl hydroquinone, vitamin E, sesamol, guaiac resin, and methionine.

A preservative may be selected from, but is not limited to, methyl paraben, ethyl paraben, propyl paraben, benzyl alcohol, chlorobutanol, phenol, meta cresol, chloro cresol, benzoic acid, sorbic acid, thiomersal, bronopol, propylene glycol, benzylkonium chloride and benzethonium chloride.

An emulsifier may be selected from, but is not limited to, acacia, carbomer, sodium lauryl sulphate, carboxymethylcellulose sodium, carrageenan, cetyl alcohol, emulsifying wax, cholesterol, cetomacrogol, methylcellulose, poloxamer, bentonite, colloidal clays, magnesium aluminium silicate, stearic acid, hydroxypropyl cellulose, polycarbophil, saponite, starch, pectin, palmitic acid, ammonium alginate, calcium, and alginate.

A texturizer may be selected from, but is not limited to, hydrocolloids, pectins, fiber, locust bean gum, alginate, gelatin, and carrageenan.

A carrier or excipient may be selected from, but is not limited to, magnesium stearate, microcrystalline cellulose, starch (corn), silicone/titanum dioxide, colloidal silicon, stearic acid, sodium starch glycolate, gelatin, a fat soluble carrier, and talc.

A composition may be provided in any suitable form, for example a tablet, capsule, oral powder, granules, liquid, beverage, injectable, patch, sticker, cream, gel, spray, sub-lingual tab, buccal tablet, pessary, enema, suppository, candy, jellies, gummy bears, varnish, baked product or edible, chewing gum, or an emulsion.

### Administration

Administering includes any mode of administration, for example oral, subcutaneous, transmucosal, transdermal, intravenous, intra-arterial, buccal, topical, rectal, vaginal, nasal, intramuscular, sublingual, inhaled. Suitably, a cannabinoid is administered orally. When administered in combination with a second or further therapeutic agent, the mode of administration of the two or more compounds may be the same or may be different.

Administration refers to delivering a cannabinoid to a subject. Administration may comprise the delivery of a single cannabinoid, as defined herein, or of two or more cannabinoids, optionally in combination with another non-cannabinoid therapeutic agent. Where two or more cannabinoids and/or therapeutic agents are administered, the administration may be separate or combined. Where separate, the administration may be simultaneous or sequential. For example, where the cannabinoids are CBD and THC, they may be delivered as a combined agent, or as separate agents either simultaneously or sequentially. Where two or more cannabinoids and/or therapeutic agents are to administered together, they may be co-formulated into a single dosage form.

Suitably, in the present invention, a cannabinoid is administered orally. Suitably, CBD optionally in combination with THC is administered orally. Suitably, the CBD is in powdered form and THC is in the form of a resin.

A cannabinoid may be administered to a subject at any suitable time. Administration may be regular, for example hourly, daily, 1 to 6 doses per day, weekly, monthly. When administered hourly or daily, this may be for a defined period of time, for example a specified number of days, a week, a fortnight, a month, or a year or more. Alternatively, administration may be before, during, or after an event or episode of a symptom or an event which triggers a symptom. Suitably, the cannabinoid is CBD, optionally in combination with THC, and is administered daily.

### Treatment

By treatment is meant an approach whose objective is to provide a beneficial effect with regard to a particular symptom, for example delayed onset of a symptom, shorter duration of the symptom, reduced severity of the symptom, reduced frequency of the symptom, a reduction in the number of symptoms. Treatment may have a beneficial effect in relation to any one or more of the symptoms or combination of symptoms disclosed herein associated with asd, increased gastrointestinal permeability and/or gut inflammation. Treatment may reduce or prevent colonic muscle thinning or increase colonic muscle thickness.

By prevention is meant means providing any treatment which causes the condition or disease, or any one or more symptoms thereof, not to develop or not to develop further. Preventative therapy may be provided to a subject who is at risk of developing the disease or condition. Prevention may inhibit or stop the onset of one or more of the symptoms or combination of symptoms disclosed herein associated with ASD, increased gastrointestinal permeability and/or gastrointestinal inflammation. Prevention may inhibit the onset or progress of colonic muscle thinning.

By amelioration is meant a reduction in the frequency, severity or time to onset of one or more symptoms. Amelioration may be defined as partial or complete relief from a symptom.

A therapeutically effective amount, or an effective amount, is an amount which has a therapeutically beneficial effect when administered to a subject, in terms of being sufficient to treat (ameliorate, reduce, eliminate or prevent) a symptom of a disease or condition. A therapeutically effective amount may vary depending upon the cannabinoid being administered, and on whether a cannabinoid is used in combination with another therapeutic agent. A therapeutically effective amount will be dependent on factors such as sex, age, weight, dosage frequency, severity of the symptom, disease or condition to be treated, the nature of the cannabinoid, the mode of administration and/or the formulation. A suitable therapeutically effective amount may be determined by a person skilled in the art using the disclosure herein, with routine experimentation.

A beneficial effect of the treatment of the present invention may be one or more of decreased gastrointestinal permeability, decreased gastrointestinal inflammation, increased or maintenance of a healthy intestinal muscle thickness, reduction in duration or frequency or severity of constipation, reduction in duration or frequency or severity of diarrhea, increased appetite, increased bladder control. A beneficial effect of the present invention may be the reduction, prevention, delayed onset, or amelioration in one or more of restlessness, aggression, rage, agitation, self-injury, tantrums, stress, anxiety, irritability, communication problems, defiance, disruptive behaviours, psychosis, lethargy, seizures, hypersensitivity, inattentiveness, stereotyped or repetitive behaviours, speech problems, sleep problems, social deficits, insomnia, learning difficulties, daily functioning difficulties, cognitive impairment or delay, attention disorder, depression, lack of concentration, dyslexia, epilepsy, motor tics, and/or vocal tics, or a gastrointestinal disorder symptoms may be selected from abdominal pain or discomfort, bloating, constipation, flatulence, lack of appetite/weight loss, incontinence, fussy eating, or diarrhoea.

Suitably, the present invention provides a reduction in irritability in a subject. Therefore, suitably, the present invention provides the use of a cannabinoid, suitably CBD optionally in combination with THC, for the treatment, amelioration or prevention of irritability in a subject diagnosed with, suffering from one or more symptoms of, or at risk of having ASD. The cannabinoid may reduce or prevent gastrointestinal hyperpermeability. The subject may have been diagnosed as having, or may be susceptible to, gastrointestinal hyperpermeability.

Suitably, the present invention provides a reduction in aggressive response in a subject. Therefore, suitably, the present invention provides the use of a cannabinoid, suitably CBD optionally in combination with THC, for the treatment, amelioration or prevention of aggressive response in a subject diagnosed with, suffering from one or more symptoms of, or at risk of having autism spectrum disorder. The cannabinoid may reduce or prevent gastrointestinal hyperpermeability. The subject may have been diagnosed as having, or may be susceptible to, gastrointestinal hyperpermeability.

Suitably, the present invention provides a reduction in hyperactivity in a subject. Therefore, suitably, the present invention provides the use of a cannabinoid, suitably CBD optionally in combination with THC, for the treatment, amelioration or prevention of hyperactivity in a subject diagnosed with, suffering from one or more symptoms of, or at risk of having ASD. The cannabinoid may reduce or prevent gastrointestinal hyperpermeability. The subject may have been diagnosed as having, or may be susceptible to, gastrointestinal hyperpermeability.

Suitably, the present invention provides an improvement in social behavior in a subject. Therefore, suitably, the present invention provides the use of a cannabinoid, suitably CBD optionally in combination with THC, for the treatment, or improvement in social behavior in a subject diagnosed with, suffering from one or more symptoms of, or at risk of having ASD. The cannabinoid may reduce or prevent gastrointestinal hyperpermeability. The subject may have been diagnosed as having, or may be susceptible to, gastrointestinal hyperpermeability.

Suitably, the present invention provides an improvement in exploratory behavior in a subject. Therefore, suitably, the present invention provides the use of a cannabinoid, suitably CBD optionally in combination with THC, for the treatment, or improvement in exploratory behavior in a subject diagnosed with, suffering from one or more symptoms of, or at risk of having ASD. The cannabinoid may reduce or prevent gastrointestinal hyperpermeability. The subject may have been diagnosed as having, or may be susceptible to, gastrointestinal hyperpermeability.

Suitably, the present invention provides a reduction in stereotypic behaviour in a subject. Therefore, suitably, the present invention provides the use of a cannabinoid, suitably CBD optionally in combination with THC, for the treatment, amelioration or prevention of stereotypic behaviour in a subject diagnosed with, suffering from one or more symptoms of, or at risk of having ASD. The cannabinoid may reduce or prevent gastrointestinal hyperpermeability. The subject may have been diagnosed as having, or may be susceptible to, gastrointestinal hyperpermeability.

### Monitoring

The present invention may provide for monitoring a subject for improvement in one or more symptoms of ASD, gastrointestinal hyperpermeability, Crohn's disease, celiac disease, ulcerative colitis, diabetes type 1, arthritis including spondylarthrites and psoriatic arthritis.

A change in a symptom may be compared to a healthy subject, or a subject known not to have or suffer from the symptom, disease or condition being monitored.

Monitoring may use any suitable method for assessing the status or progression of the symptom, disease or condition. Tests for evaluating symptoms of ASD, gastrointestinal permeability, and gastrointestinal inflammation include scans, such as an MRI scan, or testing for inflammatory agents such as acute phase proteins. Other suitable tests will be available to a person skilled in the art.

Monitoring may be conducted at any suitable time interval, for example hourly, daily, 1 to 6 times a day, weekly, fortnightly, monthly or annually.

### Diagnosis

A method of the present invention may comprise diagnosing a subject for the disease or condition, or determining susceptibility or risk of developing the disease or condition. The disease or condition may be ASD, increased gastrointestinal permeability, gastrointestinal inflammation, Crohn's disease, celiac disease, ulcerative colitis, arthritis including spondyloarthritis and psoratic arthritis. Suitably, a method of the invention comprises testing a subject for ASD, gastrointestinal permeability and/or increased gastrointestinal permeability. Suitably, a method of the invention comprises testing a subject for ASD and/or increased gastrointestinal permeability. The method may further comprise diagnosing the subject as having the disease or condition based on the test results.

Any suitable test as described herein or available in the art may be used to test for gastrointestinal hyperpermeability.

Any suitable test as described herein or available in the art may be used to test for ASD.

### Subject

The term “subject” may be used herein interchangeably with the term “patient”. A subject to be treated in the present invention may be an animal, suitably a mammal and suitably a human. A subject may be an adult or may be a child. A subject may have been, or will be, the subject of a diagnosis, treatment or observation. A subject to be treated in the present invention may have been diagnosed as having increased gastrointestinal permeability. A subject to be treated in the present invention may have been diagnosed as having ASD. Thus, a subject to be treated may have one or more behavioural symptoms and/or one or more gastrointestinal disorder symptoms. Thus, a subject to be treated may have one or more symptoms selected from the group consisting of restlessness, aggression, rage, agitation, self-injury, tantrums, stress, anxiety, irritability, communication problems, defiance, disruptive behaviours, psychosis, lethargy, seizures, hypersensitivity, inattentiveness, stereotyped or repetitive behaviours, speech problems, sleep problems, social deficits, insomnia, learning difficulties, daily functioning difficulties, cognitive impairment or delay, attention disorder, depression, lack of concentration, dyslexia, epilepsy, motor tics, and/or vocal tics. Gastrointestinal disorder symptoms may be selected from abdominal pain or discomfort, bloating, constipation, flatulence, lack of appetite/weight loss, incontinence, fussy eating, or diarrhoea. Thus, the methods and uses described herein may be useful in human therapy, or veterinary applications.

A subject to be treated in the present invention may have been diagnosed as having increased gastrointestinal permeability and/or ASD, or may be suspected of having increased gastrointestinal permeability and/or ASD, or may be at risk of developing increased gastrointestinal permeability and/or ASD. A subject to be treated may have one or more symptons of increased gastrointestinal permeability and/or ASD.

A subject to bne treated may have been diagnosed as having impaired colonic muscle thickness, or may be suspected of having impaired colonic muscle thickness or may be at risk of developing impaired colonic muscle thickness. A subject to be treated may have one or more symptons of impaired colonic muscle thickness.

A subject to be treated may have been diagnosed as having, or may be suspected of having, or may be at risk of developing a condition associated with increased gastrointestinal permeability, for example Crohn's disease, celiac disease, ulcerative colitis, diabetes type 1, arthritis including spondyloarthritis and psoriatic arthritis. A subject to be treated may have one or more symptons of such a condition.

A healthy subject may be one who does not have a gastrointestinal disorder, symptoms of a gastrointestinal disorder, a neurological disorder, symptoms of a neurological disorder, a behavioral disorder, symptoms of a behavioral disorder. A neurotypical subject suitably does not have, or is not suspected of having or predisposed to ASD.

### Dosage

The therapeutic agent may be administered at a suitable dose to achieve a beneficial therapeutic effect.

Suitable doses based on corresponding animal doses may be determined using calculations known and available in the art to determine a Human Equivalent Dose (HED). Thed conversion from animal dosages to human dosages is based upon body surface area, i.e. a mg/kg dose. A HED may be identified as a safe starting point (or initial dose) for determining an optimal dose. The HED doses referred to herein may be either a dose for administration to the subject, or may be a starting dose for further investigation. A HED may be calculated according to the guidance set out in Guidance for Industry “Estimating the Maximum Safe Starting Dose in Initial Clinical Trials for Therapeutics in Adult Healthy Volunteers” US Dept of Health and Human Services, FDA, Centre for Drug Evaluation and Research July 2005 Pharmacology and Toxicology.

A suitable human dosage may be about 0.1 to 20 mg/kg, suitably 0.5 to 15 mg/kg, suitably 0.8 to 12 mg/kg, suitably 1 to 10 mg/kg, or suitably may be about 0.1, 0.2, 0.3, 0.4, 0.5, 0.6, 0.7, 0.8, 0.9, 1, 1.5, 2, 2.5, 3, 3.5, 4, 4.5, 6, 6.5, 7, 7.5, 8, 8.5, 9, 9.5, 10, 10.5, 11, 12, 13, 14, 15, 16, 17, 18, 19 or 20 mg/kg, any range formed from any of the aforementioned integers as lower and upper limits, or any integer which falls in any of the aforementioned ranges. Most suitably, a suitable dose may be 1.6, 4.8 & 9.6 mg/kg of body weight of CBD.

Where a second therapeutic agent is co-administered with a cannabinoid, for example a second cannabinoid or THC, a suitable dose of the second therapeutic agent for a human subject may range from 0.05 to 6 mg/kg, suitably 0.1 to 5 mg/kg, most suitably 0.1 to 4 mg/kg body weight. A suitable human dosage may be about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5 or 6 mg/kg, any range formed from any of the aforementioned integers as lower and upper limits, or any integer which falls in any of the aforementioned ranges.

Suitably, where a cannabinoid and THC are used in combination as a therapy, the dose of each may be adjusted accordingly. For example, a sub-optimal dose of a first therapeutic agent may be used in combination with a sub-optimal dose of a second therapeutic agent.

Alternatively, a suboptimal dose of a first therapeutic agent may be used in combination with an optimal dose of a second therapeutic agent. Alternatively, an optimal dose of a first therapeutic agent may be used in combination with an sub-optimal dose of a second therapeutic agent. Where for example the first therapeutic agent is CBD and the second therapeutic agent is THC, the CBD may be administered in a sub-optimal dose and the THC may be administered in a sub-optimal or optimal dose. For example, CBD may be administered at about 10-60 mg/kg, suitably about 20-40 mg/kg, suitably about 20, 30 or 40 mg/kg and THC may be administered at about 0.1 to 5 mg/kg.

Where a cannabinoid and THC are used in combination as a therapy, the amount of each present in a combined or separate dosage may be in a ratio of 1:1 to 50:1 CBD to THC, 5:1 to 40:1, suitably 5:1 to 20:1, most suitably 10:1. Suitably, the ratio is 10:1 CBD to THC in a granular composition comprising 45% CBD/THC.

### Methods of Treatment

Also provided is a method of preventing or treating gastrointestinal hyperpermeability in a subject, wherein the method comprises administering a cannabinoid to the subject. Suitably, the method comprises diagnosing the subject with ASD.

Also provided is a method of preventing or treating a symptom of ASD in a subject, wherein the method comprises administering a cannabinoid to the subject, wherein the subject has been diagnosed as having gastrointestinal hyperpermeability.

Also provided is a method of preventing or treating gastrointestinal inflammation in a subject, wherein the method comprises administering a cannabinoid to the subject.

Suitably, a method of treatment comprises a diagnosis of the subject. A method of the invention may comprise testing a subject for presence or risk of gastrointestinal hyperpermeability, or ASD, or any disease or condition as described herein. A method of the invention may comprise analysing the results of a test to provide a diagnosis. A diagnosis may provide an indication or presence, absence, risk or severity of a disease or condition as disclosed herein.

The embodiments and features described herein with reference to the use of a cannabinoid for treatment apply mutatis mutandis to other aspects of the invention, for example the methods of treatment as described herein.

The term about in relation to a numerical value or range means up to and including +/−10% of the numerical value or range.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

In rodents, given the lack of effect of thalidomide, the administration of VPA at the time of neural tube closure on gestational day 12.5 results in brain abnormalities resembling those found at autopsy and in brain-imaging studies of patients with autism including cell loss and morphological abnormalities in the cerebellum (Ingram et al., 2000). Moreover, the offspring of VPA exposed animals exhibit an array of developmental and behavioural deficits corresponding to those observed in autism. Principle amongst these are altered sensitivity to stimulus, sensorimotor gating, stereotypical behaviours, cognitive deficits and social dysfunction (Schneider and Przewlocki, 2005; Wagner et al., 2006). Play behaviour is highly expressed in rodent species but is particularly evident during the juvenile stage of life. Such play is highly ritualised and incorporates many behaviours observed in adult life, yet these are distinct differences, for example aggression in adult rats is typically noted in biting of back and hindquarters whereas in play pairs focus on nose to neck thrusting accompanied by mounting and pinning that is of short duration (Pellis et al., 1997). Social play has a considerable incentive value and is important for the development of social skills and the selection of appropriate behavioural patterns (Vanderschuren et al., 1997). The onset of play fighting begins at about 18 days postnatal and becomes maximal between days 30 and 40 decreasing post-puberty (Pellis et al., 1997). This contrasts with adult aggressive behaviour that becomes more evident following puberty and is expressed in a hierarchical dominant manner within the rodent environment. Irritability behaviour can be measured in rodents using the bottlebrush task (Kononoff et al., 2018) and has been assessed in previous in-house studies utilising the rat VPA model with relevance to ASD.

The similarities in anatomic pathology in patients and offspring of VPA rats support the use of the in utero VPA rodent model. Significantly, the availability of an animal model expressing the neuropathological deficits enables the detailed evaluation of the effects of novel therapeutics on the core features, associated symptoms and irritability behaviour in these diseases.

The offspring of valproic acid (VPA) exposed animals exhibit an array of developmental and behavioural deficits with relevance to autism spectrum disorders (ASD). The aim of this study was to evaluate the potential therapeutic action of cannabidiol (CBD) alone and in combination with tetrahydrocannabinol (THC), to ameliorate gastrointestinal and behavioural deficits in the male offspring of female rats given valproic acid (VPA) during pregnancy.

Time-mated female Wistar rats were administered a single dose of valproic acid (VPA, 600 mg/kg, i.p.) on gestational day 12.5 (VPA/VEH). To determine behavioural deficits in the VPA autism model, male offspring were tested at juvenile (postnatal day 30) for the presence of abnormal behaviours associated with autism e.g., decreased sociability and increased irritability.

Two experimental cohorts were prepared, the first to evaluate CBD alone and the second to evaluate a sub-optimal dose of CBD in combination with increasing THC dose.

In the first study, a sub-chronic 8-day dosing schedule was employed with the test compound CBD administered alone (10, 30 and 60 mg/kg, p.o. VPA/CBD; dose volume 5 ml/kg). Control animals were administered sterile saline (0.9% w/v, i.p.) in utero and the vehicle control (ethanol: Kolliphor HS-15:0.9% saline (1:1:18, v/v/v) p.o. VEH/VEH). The test compound and vehicle control were administered to the animals once daily for 7 days prior to, and once again on each day of behavioural assessment, 60 minutes prior to test which consisted of a battery of behavioural paradigms: P30 social play and bottle brush test, P31-32 open field exploratory behaviour. A reference positive control, the atypical antipsychotic risperidone (0.125 mg/kg, i.p., once per day for 8 days prior to and for duration of behavioural testing), was included in study one for comparison. Subsequent to behavioural analysis the effect of CBD on gastrointestinal permeability was evaluated four hours following oral administration of fluorescein isothiocyanate-labelled dextran measured in plasma on postnatal day 33. On postnatal day 34, 60 minutes post a final treatment with the test agents, animals were humanely euthanised and samples collected to evaluate inflammatory biomarkers, gastrointestinal function and histology. 5 In the second study, the sub-optimal dose of CBD (30 mg/kg, p.o.) was evaluated in combination with administration of THC at increasing concentration (0.75, 1.5 and 3 mg/kg, p.o. VPA/CBD+THC). Comparison was made to CBD administered alone at the sub-optimal and effective doses (30 and 60 mg/kg, p.o.) and to vehicle controls as per study one. Behavioural and gastrointestinal readouts were examined as per study one with terminal samples collected on the final day of the experiment.

TABLE 1
Summary table
Cohort one—CBD alone

| | CBD alone | | | Risperidone |
|---|---|---|---|---|
| Readout | 10 mg/kg | 30 mg/kg | 60 mg/kg | 0.125 mg/kg |
| play behaviour | Ø | Ø | + | Ø |
| bottle brush | Ø | + | + | + |
| irritability | | | | |
| open field | Ø | Ø | + | + |
| (spontaneous | | | | |
| LMA on day 1) | | | | |
| open field | Ø | Ø | + | + |
| (habituation | | | | |
| LMA on day 2) | | | | |
| centre crossing | Ø | Ø | Ø | Ø |
| centre time | Ø | Ø | Ø | Ø |
| Circling | Ø | Ø | + | Ø |
| Grooming | Ø | Ø | + | Ø |
| gastrointestinal | Ø | + | + | Ø |
| permeability | | | | |
| MPO activity | Ø | Ø | + | Ø |

(+: improvement, Ø: no change)
Abbreviations:
CBD—cannabidiol;
LMA—locomotor activity;
MPO—myeloperoxidase.

TABLE 2
Summary table
Cohort two—CBD alone and in combination with THC

| | CBD | CBD | CBD | CBD | CBD |
|---|---|---|---|---|---|
| | (60 mg/kg) | (30 mg/kg) | (30 mg/kg) | (30 mg/kg) | (30 mg/kg) |
| | THC | THC | THC | THC | THC |
| Readout | (0 mg/kg) | (0 mg/kg) | (0.75 mg/kg) | (1.5 mg/kg) | (3 mg/kg) |
| play behaviour | + | Ø | Ø | Ø | + |
| bottle brush | + | Ø | Ø | Ø | + |
| irritability | | | | | |
| open field | + | Ø | Ø | Ø | + |
| (spontaneous | | | | | |
| LMA on day 1) | | | | | |
| open field | + | Ø | Ø | Ø | + |
| (habituation | | | | | |
| LMA on day 2) | | | | | |
| centre crossing | + | Ø | Ø | Ø | Ø |
| centre time | + | Ø | Ø | Ø | Ø |
| Circling | + | + | + | Ø | + |
| grooming | + | Ø | Ø | Ø | + |
| gastrointestinal | + | Ø | Ø | + | + |
| permeability | | | | | |
| MPO activity | + | Ø | Ø | + | + |

(+: improvement, Ø: no change)
Abbreviations:
CBD—cannabidiol;
THX—tetrahydrocannabinol,
LMA—locomotor activity;
MPO—myeloperoxidase

The results of this study indicate that the behavioural effects generated by in utero VPA exposure are reproducible changes in social preference, hyperactivity, stereotypy and irritability. Furthermore, the VPA autism model exhibits markers for altered gastrointestinal function that correlate to clinical presentations by autism patients. The aforementioned behavioural and gastrointestinal changes are improved by the administration of the test doses of CBD, with further evidence of additive benefit with the combination treatment of CBD and THC together.

This study evaluated the potential therapeutic action of CBD alone and in combination with THC to ameliorate gastrointestinal and behavioural deficits in the male offspring of female rats given valproic acid (VPA) during pregnancy. The VPA model is an established model of autism (Schneider and Przewlocki, 2005).

Two experimental studies carried out 1). that established the effective dose of CBD and 2). determined the additive effect of CBD in combination with THC at increasing ratios. To achieve this, we conducted the following readouts for each experiment:
exposure of time mated female Wistar rats to VPA on day 12.5 of gestation to induce a developmental model of autism. Offspring from vehicle-treated controls were reared in parallel;
after dosing from day 23 the effect of the test treatments compared with vehicle (n=8 per group), was determined on abnormal social play following VPA exposure at postnatal day 30;
determined the effect of the test treatments compared with vehicle (n=8 per group) on irritability in the bottle brush test at postnatal day 30;
determined the effect of the test treatments compared with vehicle (n=8 per group) on hyperactivity and stereotypical behaviour in an open field arena at postnatal days 31 and 32;
determined the effect of the test treatments compared with vehicle (n=8 per group) on gastrointestinal permeability four hours following oral administration of fluorescein isothiocyanate-labelled dextran measured in plasma on postnatal day 33;
determined the effect of the test treatments compared with vehicle (n=8 per group) on gastrointestinal dysfunction employing intestinal myeloperoxidase activity in homogenized samples of distal colon collected on postnatal day 34;
completed collection and preservation of caecum microbiome samples on postnatal day 34 for subsequent determination of the effect of the test treatments compared with vehicle (n=8 per group) on gastrointestinal microbiota;
completed collection and preservation of ileum and colon samples on postnatal day 34 for subsequent determination of the effect of the test treatments compared with vehicle (n=8 per group) on gastrointestinal histology;
investigated colonic muscle thickness in valproic acid rodent model of autism treated with test compounds
completed collection and preservation of plasma, brain and ileum samples on postnatal day 34 for subsequent determination of the effect of the test treatments compared with vehicle (n=8 per group) on inflammatory cytokine expression;
completed collection and preservation of plasma samples on postnatal day 34 for subsequent determination of the plasma level of the test treatments (n=8 per group);
In accordance with the protocol (below), for study one CBD at three doses was compared to vehicle control, in addition to in utero vehicle control and reference control groups (n=8).

For study two, the suboptimal dose (30 mg/kg) of CBD was evaluated in combination with increasing ratio of THC (0.3, 1 and 3 mg/kg p.o.) compared to CBD alone at sub-optimal and effective doses (30 and 60 mg/kg), vehicle control, in addition to in utero vehicle control (n=8).

TABLE 3
Study groups

| Study 1—efficacy | Study 2—combination |
|---|---|
| Vehicle/vehicle | Vehicle/vehicle |
| VPA/vehicle | VPA/vehicle |
| VPA/CBD (10 mg/kg) | VPA/CBD (30 mg/kg) |
| VPA/CBD (30 mg/kg) | VPA/CBD (60 mg/kg) |
| VPA/CBD (60 mg/kg) | VPA/CBD (30 mg/kg) + THC |
| VPA/risperidone (0.125 mg/kg) | (0.75 mg/kg) |
| | VPA/CBD (30 mg/kg) + THC |
| | (1.5 mg/kg) |
| | VPA/CBD (30 mg/kg) + THC (3 mg/kg) |
| 6 groups, n = 8, 48 animals | 7 groups, n = 8, 56 animals |

### Experimental Procedure

### 1 Animals

### 2 in Utero Valproic Acid Exposure

Time-mated female Wistar Han Rcc rats (Envigo, UK) were administered a single dose of valproic acid (VPA, Sigma Aldrich, Ireland, lot no. MKCB2699V, 600 mg/kg, i.p.) or vehicle (0.9% saline i.p.) on gestational day 12.5. Confirmation of the VPA induced developmental deficit was rapidly achieved by examination of characteristic “tail kinks” observed in all of the juvenile animals exposed to VPA. Litters were normalised to 8 animals and to male offspring at postnatal day 3. Male pups were cross fostered to make up the uniform litter size of eight. Male offspring were housed as per standard laboratory conditions until time of experimental use. Vehicle-treated control animals were employed as controls, with animals for both parts of the study prepared together. At weaning, animals were allocated into test groups according to a balanced design based on weight and litter to distribute these equally across each treatment group. Animals were introduced to the experimental holding rooms prior to the commencement of the study, housed in groups of 4 during this period in soft bottomed cages with sawdust bedding material, with enrichment consisting of nesting material, cardboard tubes and Perspex internal shelter, and maintained at 22-24° C. on a standard 12-hour light/dark cycle (07.30-19.30), with food and water available ad libitum. All animals were examined and weighed daily during drug treatment and experimental protocols. All procedures were carried out by individuals retaining the appropriate authorisation from the Irish Government Health Products Regulatory Authority. Furthermore, the study protocol was approved by the Animal Research Ethics Committee of University College Dublin, Ireland.

### Compound Formulation and Administration

### Preparation of CBD and THC

Doses (10, 30 and 60 mg/kg, p.o.) of CBD (batch number LL77122021, CB21 Pharma, Czech Republic) and (0.75, 1.5 and 3 mg/kg, p.o.) THC (batch number K745, Purisys, USA) were selected by the sponsor based in anticipated human therapeutic dose-level and the results obtained from previous animal studies. The agents were provided as pure compounds, as powdered CBD and THC resin. Certificate of analysis for each is attached to Appendix B. For administration of CBD alone or in combination with THC these were formulated at the required concentration in a vehicle of ethanol: Kolliphor HS-15:0.9% saline (1:1:18, v/v/v). The following steps were employed:
1. Heat required amount of Kolliphor HS-15 and Saline (separately) to 60° C. in an oven. Also place the required volumetric flask into the oven. Heat items for at least 30 mins and ensure the Kolliphor HS-15 has melted.
2. Weigh the required amount of Ethanol into a glass beaker/bottle, add the required amount of CBD and/or THC and agitate until dissolved.
3. Weigh the required amount of Kolliphor HS-15 into the beaker/bottle containing the CBD and/or THC Ethanol mix.
4. Place a stirrer bar into the beaker/bottle and place onto a hotplate magnetic stirrer, mix the mixture and control the temperature of the mixture at 60° C.
5. Once the mixture is homogenous add half of the required final volume of hot Saline to the glass beaker/bottle while mixing using the hotplate stirrer.
6. Once the solution is homogenous transfer the solution into the pre-heated volumetric flask.
7. Add half of the remaining volume of Saline to the now empty glass beaker/bottle and stir to incorporate any residue and add to the volumetric flask, repeat this step a further time.
8. Make up to the mark using Saline.
9. Invert the volumetric flask until the solution is homogenous.
10. Protect from light and ensure the solution has cooled prior to dosing.
11. Keep solution on a magnetic stirrer for duration of dosing.

Fresh formulation was prepared every 4 days and was kept in a fridge at 4° C. protected from light once dosing was complete on each day. Each dose was formulated separately. Solutions from the fridge were allowed to reach room temperature and vortexed prior to dosing and maintained on a magnetic stirrer during dosing. A dose volume of 5 ml/kg via oral gavage was employed.

### Preparation of Vehicle

The vehicle group was dosed orally with the vehicle used to dissolve CBD alone or in combination with THC, namely ethanol: Kolliphor HS-15:0.9% saline (1:1:18, v/v/v). Administered via oral gavage at a dose volume of 5 ml/kg.

### Preparation of Risperidone

Risperidone (Merck, UK) was used as a clinical reference control (0.125 mg/kg, i.p.) in post weaning behavioural tests. Volume of injection was 1 ml/kg prepared in 0.9% saline (w/v). The required amount of risperidone was weighted out in a glass beaker and dissolved in a drop of 10% glacial acetic acid. The solution was brought to volume with 0.9% saline (w/v) and the pH adjusted to ~6.8 with 2M NaOH. Fresh solutions were prepared every two days.

### Administration of Test Formulations

Two experimental cohorts were prepared, the first to evaluate CBD alone and the second to evaluate a sub-optimal dose of CBD in combination with increasing THC dose.

### Efficacy Study

The treatment regimen employed an 8-day dosing schedule prior to behavioural readout for three dose groups of CBD (10, 30 and 60 mg/kg) oral gavage. Volume of injection was 5 ml/kg. CBD was administered daily between postnatal days 23 and 34. Drug administration continued for each day of behavioural assessment and gastrointestinal activity measurement, administered 60 minutes prior to test. In each case, vehicle treated controls were employed for comparison. Moreover, the atypical antipsychotic risperidone, which currently has a label to treat irritability in autism, was used as a clinical reference control (0.125 mg/kg i.p., once per day between postnatal days 23 and 34; dose volume=1 ml/kg).

TABLE 4
CBD efficacy study

| | | | Pre- | P30 - | P30 - | | P33 - | P34 - |
|---|---|---|---|---|---|---|---|---|
| | Prenatal | Postnatal | treatment | social | bottle | P31-32- | Gastrointestinal | sample |
| Group | treat [symbol] | treatment | time | play | brush | LMA | [symbol] | collection |
| Group | Vehicle | Vehicle | 60 mins | 4 pairs | 8 | 8 | 8 | 8 |
| A | | | | | | | | |
| Group | VPA | Vehicle | 60 mins | 4 pairs | 8 | 8 | 8 | 8 |
| B | | | | | | | | |
| Group | VPA | CBD | 60 mins | 4 pairs | 8 | 8 | 8 | 8 |
| C | | (10 mg/kg) | | | | | | |
| Group | VPA | CBD | 60 mins | 4 pairs | 8 | 8 | 8 | 8 |
| D | | (30 mg/kg) | | | | | | |
| Group | VPA | CBD | 60 mins | 4 pairs | 8 | 8 | 8 | 8 |
| E | | (60 mg/kg) | | | | | | |
| Group | VPA | Risperidone | 60 mins | 4 pairs | 8 | 8 | 8 | 8 |
| F | | (0.125 mg/ | | | | | | |
| | | [symbol] | | | | | | |

| | | | | | |
|---|---|---|---|---|---|
| Total | 24 pairs | 48 | 48 | 48 | 48 |

[symbol]

indicates data missing or illegible when filed

### Combination Study

The treatment regimen was an 8-day dosing schedule prior to behavioural readout for the suboptimal dose of CBD (30 mg/kg, confirmed in study one) via oral gavage, alone and in combination with increasing ratio of THC (0.75, 1.5 and 3 mg/kg). Volume of injection was 5 ml/kg. The test agents will be administered daily between postnatal days 23 and 34. Drug administration continued for each day of behavioural assessment and gastrointestinal activity measurement, administered 60 minutes pre-test as a single preparation. In each case, vehicle treated controls and the effective dose of 60 mg/kg CBD alone were employed for comparison.

TABLE 5
CBD and THC combination study

| | | | Pre- | P30 - | P30 - | | P33 - | P34 - |
|---|---|---|---|---|---|---|---|---|
| | Prenatal | Postnatal | treatment | social | bottle | P31-32 - | Gastrointestinal | sample |
| Groups | treatme [symbol] | treatment | time | play | brush | LMA | [symbol] | collectio [symbol] |
| Group G | Vehicle | Vehicle | 60 mins | 4 pairs | 8 | 8 | 8 | 8 |
| Group H | VPA | Vehicle | 60 mins | 4 pairs | 8 | 8 | 8 | 8 |
| Group I | VPA | CBD | 60 mins | 4 pairs | 8 | 8 | 8 | 8 |
| | | (30 mg/kg) | | | | | | |
| Group J | VPA | CBD | 60 mins | 4 pairs | 8 | 8 | 8 | 8 |
| | | (60 mg/kg) | | | | | | |
| Group K | VPA | CBD | 60 mins | 4 pairs | 8 | 8 | 8 | 8 |
| | | (30 mg/kg) + | | | | | | |
| | | THC | | | | | | |
| | | (0.75 mg/k [symbol] | | | | | | |
| Group L | VPA | CBD | 60 mins | 4 pairs | 8 | 8 | 8 | 8 |
| | | (30 mg/kg) + | | | | | | |
| | | THC | | | | | | |
| | | (1.5 mg/kg [symbol] | | | | | | |
| Group M | VPA | CBD | 60 mins | 4 pairs | 8 | 8 | 8 | 8 |
| | | (30 mg/kg) + | | | | | | |
| | | THC | | | | | | |
| | | (3 mg/kg) | | | | | | |

| | | | | | |
|---|---|---|---|---|---|
| Total | 28 pairs | 56 | 56 | 4 pairs | 56 |

Total study animal number: 48 + 56 = 104 [symbol]
indicates data missing or illegible when filed

Animals were assessed in each paradigm as outlined below. All animals were tested in the behavioural tasks on the morning of the same training day. To reduce potential circadian bias each treatment group was trained evenly over the course of the morning with all assessments concluded before midday.

### Behavioural Endpoints

### Social Play (Play)—(P30)

Play behaviour is highly expressed in rodent species but is particularly evident during the juvenile stage of life. Such play is highly ritualised and incorporates many behaviours observed in adult life, yet there are distinct differences, for example aggression in adult rats is typically noted in biting of back and hindquarters whereas in play pairs focus on nose to neck thrusting accompanied by mounting and pinning that is of short duration (Pellis et al., 1996). Such characteristic play behaviour is typically maximised around postnatal days 25 to 35 (Pellis and Pellis, 2009). Therefore, at postnatal day 30 and following 8 days treatment with the test compounds, animals were evaluated for juvenile play behaviour. This test was carried out under low light in a novel high walled open field test arena (40×40×25 cm) (1×w×h), wherein paired animals from the same treatment group, but different home cages, were examined over a 15-minute trial. Animals were first allowed to individually explore the arena for a 10-minute acclimatisation trial (P29) and 24 hours later evaluated for social play (P30). On day of evaluation animals were scored for play activity measured by pinning and mounting (latency to, frequency and total duration) and also for general social behaviour (following, sniffing, licking or social grooming of test partner). Scoring was conducted blind by manual video analysis employing Ethovision XT (Noldus, UK) software linked to an overhead video camera. With social play and direct social interaction being reciprocal behaviours, one pair of rats from the same treatment group but different home cages generated one data point. Therefore, although n=8 rats per treatment group participated in the measurements, that combined measurement means the social play/interaction analysis have a group size of n=4.

### Irritability-Like Behaviour: Bottle-Brush Test—P30

To test irritability-like behaviour we employed the bottle-brush test modified for rats (Riittinen et al. 1986). Irritability-like behaviour was examined by measuring aggressive and defensive responses during the bottle-brush test carried out in juvenile animals immediately after play behaviour evaluation at P30. Irritability-like behaviour testing was performed under dark conditions with a red light and infrared camera recording (Kononoff et al., 2018). Testing consisted of 10 trials per rat in plastic cages (377×325×170 mm) with fresh bedding. During each trial, the rat was placed at the back of the cage. A bottle-brush was rotated toward the animal's whiskers (from the front of the cage). The brush was rotated around the whiskers of the rat for approximately 1 second. The brush was then rotated back to the front of the cage where it hung vertically for approximately 2 seconds, during which behavioural responses were recorded. An intertrial interval of 10 seconds was used, with each new trial started with the animal returned to the back of the cage. All testing was conducted in the morning, starting at 09.00 and concluded before 12.00.

Employing video analysis of the test the following were scored as aggressive responses: smelling the brush, biting the brush (during the initial phase of rotating the brush forward and back to the starting position), boxing the target, following the brush, exploring the brush (using paws or mouth to manipulate the brush without biting or boxing), mounting the brush, and delayed biting (during the 2 s that the brush hung at the starting position). The following were scored as defensive responses: escaping from the brush, digging, burying, jumping, climbing, vocalization, freezing, and grooming. Grooming and digging were additionally recorded during the 10 second intertrial intervals. Frequency and total time engaged in each behaviour and the sum of aggressive and defensive behaviours, referred to as the irritability score (Kononoff et al, 2018), were recorded.

### Hyperactivity, Grooming, Stereotypy: Open Field Behaviour—P31-32

Following eight days treatment with the test compound animals were scored for locomotor activity and exploratory behaviour in two 10-minute trials within an open field arena over two days, a protocol widely used at Berand and allows to distinguish between day 1 spontaneous locomotor activity and day 2 locomotor activity following habituation (pre-exposure to the arena on day 1). Each day the animals were placed into a 64 cm² black Perspex box with 30 cm side walls, under low light and monitored via remote video capture for spontaneous activity. Linked to Ethovision XT image analysis software these videos were analysed for total distance moved and number and duration of centre zone crossings as an indication of hyperactivity and anxiety-like behaviour. Manual scoring of the videos was conducted (blinded to treatment group) to determine frequency of circling behaviour (number of 360 degree turns) and duration of self-grooming (n=8).

### Gastrointestinal Endpoints

### Gastrointestinal Permeability—P33

This assay is an indirect measure of total intestinal permeability. Animals are given an oral gavage of a fluorescently labelled small molecule (FITC-dextran), and at a subsequent timepoint the difference in fluorescence pre- and post-gavage is measured in plasma (Thevaranjan et al., 2017). Prior to gavage, a 50 μl sample of blood was collected by tail vein puncture. Animals then received 500 μl of 80 mg/ml FITC dextran (4 kDa Merck Ireland) by oral gavage and four hours later a second tail vein blood sample was collected. Tail vein blood sampling is facilitated through use of local anaesthetic EMLA cream following sterilisation of the tail. Blood samples were treated with 15% v/v acid-citrate-dextrose solution as an anticoagulant (38 mM citric acid, 107 mM sodium citrate, 136 mM dextrose), then mixed thoroughly by inversion and centrifuged at 5,000 rpm for 10 minutes. The plasma supernatant was transferred to microfuge tubes stored protected from light at 4° C. Each pre- and post-test plasma sample was diluted 1:5 in 1×PBS and a 100 μl aliquot was transferred to a 96-well plate. Readings of relative fluorescence units by a spectrophotometer were measured in duplicate and averaged. Fluorescence was determined at 530 nm with excitation at 485 nm. Permeability was expressed as relative fluorescence units between the groups being compared, determined by subtracting the PBS blank fluorescence from all samples and then subtracting the post-gavage fluorescence from the pre-gavage fluorescence. Treatment groups were prepared and analysed according to a blocked design to prevent bias.

### Terminal Sample Collection—P34

At postnatal day 34, 60 minutes post-treatment with the CBD, CBD plus THC, vehicle or risperidone formulations in each experimental study the following tissue samples were collected. Vials were labelled clearly with codes to identify study reference, date, animal ID, treatment group and tissue sample type.

### Plasma

Trunk blood was collected into labelled tubes (5 ml) containing Lithium Heparin and inverted 8-10 times to ensure homogenous distribution of Lithium Heparin and kept on ice. The tubes were centrifuged for 10 minutes at 3300 g at 4° C. within 30 minutes of collection and frozen within 30 minutes of centrifugation. The extracted plasma was then pipetted into pre-labelled 1.5 ml LoBind Eppendorf tube (1 aliquot) in two 100 μl aliquots, labelled, frozen and stored in a −80° C. freezer until shipment. One aliquot was for analysis of cytokine expression and a second for measurement of test formulation plasma levels. Samples were shipped at minus 80° C. to Transpharmation Ltd at Trinity College Dublin, Ireland (cytokine expression) and stored for a second vendor for bioanalysis.

### Caecal Sample for Microbiome Analysis

Caeca were removed intact from the euthanised animals under sterile conditions, contents extracted into sterile labelled 1.5 ml LoBind Eppendorf tubes, frozen and stored at −80° C. until shipped for analysis.

### Colon Sample Collection

Five animals were chosen at random from each of the agreed treatment groups. Colon tissue was collected after euthanasia, weighted, flushed using cold sterile saline, and divided into three sections. One distal sample (approx. 1 cm×1 cm) was employed for intestinal myeloperoxidase (MPO) activity as described below by Berand Ltd. This sample was weighed and stored at −80° C. until use. A second proximal colon sample (approx. 1 cm×5 cm) was prepared for histological analysis by collection using the Swiss roll technique, placed into a cassette for paraffin embedding and preservation in 10% formalin solution for 24 hours. They were then transferred into 70% ethanol solution until paraffin embedded. These samples were shipped in 70% ethanol solution to Transpharmation Ltd at trinity College Dublin, Ireland for further processing. Haematoxylin & eosin staining was performed on the tissue before imaging. Five images were taken from each animal at 1.25× magnification. Using ImageJ the thickness of the muscle layer was measured at 5 locations throughout the section. A third medial section of colon (approx. 1 cm×2.5 cm) was frozen in liquid nitrogen for the analysis of cytokine expression and was shipped at minus 80° C. to Transpharmation Ltd at Trinity College Dublin, Ireland.

### Haematoxylin & Eosin Staining of Tissue Sections

Staining was performed using the Haematoxylin and Eosin kit from Vector Laboratories (Cat. No. H-3502). Sections were deparaffinized and rehydrated by washing in xylene (3×5 min), 100% ethanol (1 min), 96% ethanol (1 min) and distilled water (2 min). Each section was fully covered in haematoxylin stain and left for 5 min before being washed twice in distilled water for 15 s. Bluing agent was applied to sections for 10-15 s before washing twice in distilled water for 15 s. Slides was submerged in 100% ethanol for 10 s before each section was covered with Eosin Y for 2-3 min. Slides were then submerged in 100% ethanol for 10 s, washed twice in 100% ethanol for 1 min each, and finally dehydrated in xylene (3×1 min).

Coverslips were mounted using VectaMount Mounting Medium (Vector Laboratories, Cat. No. H-5000) and left to dry overnight.

### Stained Tissue Section Imaging

Sections were imaged using an Olympus BX51 microscope at a magnification of 1.25×. Images were then analysed using ImageJ software. Thickness of muscle were measured from luminal surface to muscularis mucosa and from luminal surface to external muscle layer. This was repeated at 5 different locations across the section.

### Small Intestine Sample Collection

The small intestinal tissue was collected and preserved for future analysis. As per DeTheije et al 2014 (Brain Behav Immun 37:240) small intestinal tissue was removed, opened longitudinally, divided in a 7 cm proximal part (jejunum) and 7 cm distal part (ileum), rolled using the Swiss roll technique placed into a cassette for paraffin embedding and preservation in 10% formalin solution for 24 hours. They were then transferred into 70% ethanol solution until paraffin embedded. These samples were shipped in 70% ethanol to Transpharmation Ltd at Trinity College Dublin, Ireland for processing.

### Intestinal Myeloperoxidase (MPO) Activity

Analysis of colonic myeloperoxidase (MPO) activity employed a colorimetric activity assay kit (MAK068, Sigma-Aldrich, Ireland). Samples were analysed according to the manufacturer's instructions. Briefly, weighted distal colon tissues from all the groups were rapidly homogenized with 4 volumes of MPO Assay buffer and then centrifuged (13000 g×10 min, 4° C.) to obtain the supernatant. A 50 μl aliquot of each was added to a flat bottomed 96 well plate and brought to a final volume of 100 μl with MPO assay buffer. The MPO activity in milliunits/mL was determined using a 5,5′-dithiobis-(2-nitrobenzoic acid) (DTNB) probe to produce a TNB chromophore in a standard curve (0-50 nmol/mL). Standards and blank were prepared according to manufacturer's instruction. Absorbance was read at 412 nm at 60 minutes and expressed per mg of tissue sample. Treatment groups were processed and analysed according to a blocked design to preclude bias.

### Brain

Whole brain was collected for inflammatory cytokine measurement, divided into hemispheres and each tissue weighed, flash frozen in liquid nitrogen, stored individually in labelled tubes at −80° C. until shipment. Samples were shipped at minus 80° C. to Transpharmation Ltd at Trinity College Dublin, Ireland.

### Statistical Analysis

All data will be assessed for normality (D'Agostino and Pearson normality test or Shapiro-Wilks test for n<8 or unequal groups) and outliers (ROUT; Prism V9.2) removed accordingly and normality reassessed. Where necessary, data may be transformed to achieve normality (log 10 or square root) and analysed parametrically. Data that are non-normally distributed are analysed using Kruskal-Wallis followed by planned comparisons with Dunn's correction. Normally distributed data are analysed using one-way ANOVA followed by planned comparisons with Sidak's correction. Data from time spent in the chamber of the social approach/avoidance and bottle brush test at P60-63 will be analysed by two-way ANOVA (or repeat measure 2-way ANOVA in R if non-normally distributed) followed by Dunnett's multiple comparison test.

Data that follow a normal distribution will be presented as mean±SEM. Data that do not follow a normal distribution will be presented as box and whisker plots with individual values plotted (median and IQR, with the tails showing the highest and lowest values). Statistical significance will be defined as p<0.05.

For the histological analysis of colonic tissue, statistical analysis was performed using GraphPad Prism 9. Normality was assessed using the Shapiro-Wilk test. As data did not meet the assumption of normal distribution, a Kruskal-Wallis test followed by an uncorrected Dunn's post-hoc test were performed to compare mean values across groups. Results were considered significant if p<0.05.

### Results

### Study One—CBD Efficacy

### Social Play Behaviours—(P30)

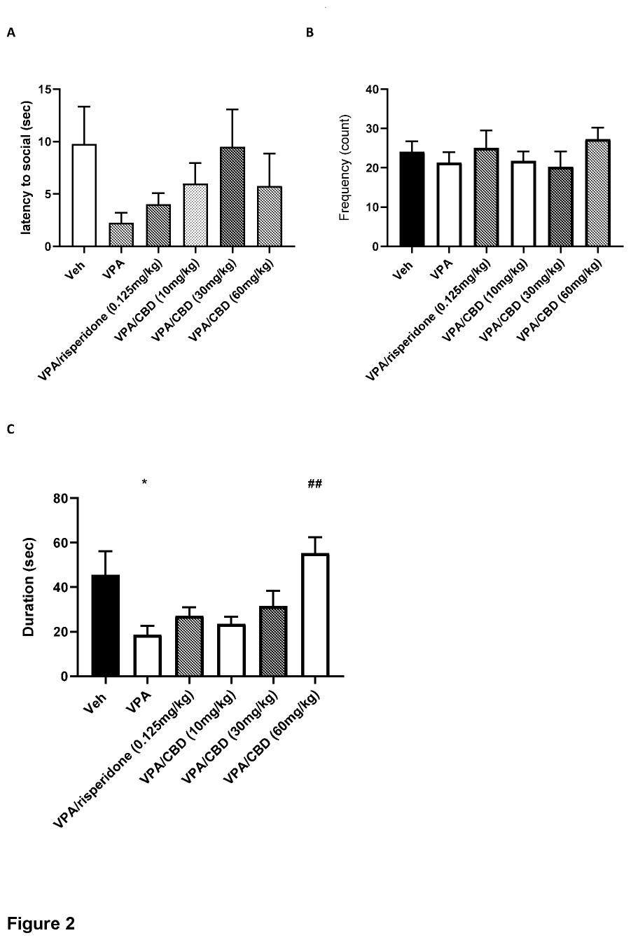

When investigating reciprocal social behaviour, one-way ANOVAs revealed no significant effects on latency to or frequency of social play (F[5, 18]=1.295 and 0.648, P=0.3095 and 0.6666, respectively) (FIGS. 2A and 2B), however there was a significant main effect on and duration of social play (F[5, 18]=4.634, P=0.0068) (FIG. 2C). Post-hoc analysis revealed significant decreases in duration of social behaviour of VPA exposed animals when compared to vehicle treated control animals (VEH/VEH vs VPA/VEH, P=0.0444). CBD at 60 mg/kg (P=0.0044) significantly attenuated the reduction in social play duration.

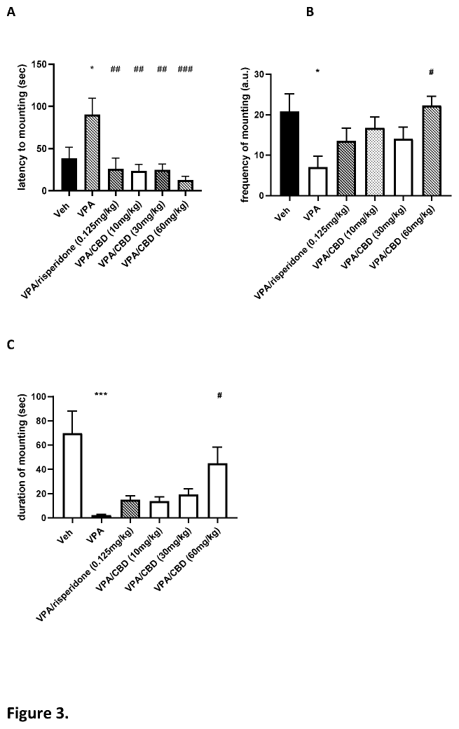

When investigating mounting behaviours, one-way ANOVA showed there was a significant main effect of treatment on latency to engage in mounting play behaviour (F[5, 18]=5.636, P=0.0027) (FIG. 3A). Post-hoc analysis revealed significant delay in onset of social mounting behaviours by VPA exposed animals when compared to vehicle treated control animals (VEH/VEH vs VPA/VEH, P=0.0293). CBD at 10, 30 and 60 mg/kg (P=0.0039, P=0.0047 and P=0.001, respectively) significantly attenuated the delay in social play. Moreover, one-way ANOVA revealed a significant main effect on the frequency and duration of mounting play (F[5, 18]=3.177, P=0.0314 and F[5, 18]=6.759, P=0.001; respectively) (FIGS. 3B and 3C). Post-hoc pairwise analysis of frequency of mounting behaviour showed a significant effect of valproic acid exposure when compared to vehicle controls (P=0.0284), a decrease ameliorated following 60 mg/kg CBD treatment (P=0.0134). Likewise, post-hoc pairwise analysis of duration of mounting behaviour revealed a significant effect of valproic acid exposure when compared to vehicle controls (P=0.0005) a decrease in play reversed following treatment with CBD at 60 mg/kg (P=0.0278).

Risperidone (0.125 mg/kg) was without effect on any other measure of play or social interaction in juvenile animals as per previous experiments.

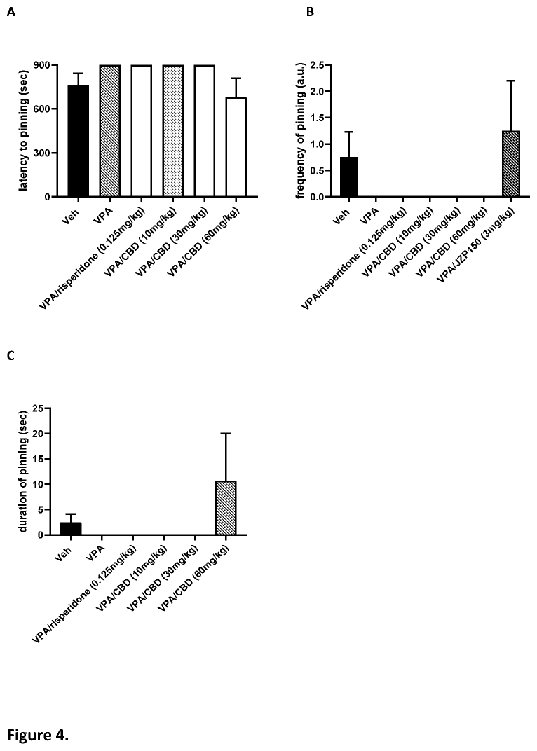

No significant effect of VPA exposure or drug treatment was evident on pinning play behaviour which overall was extremely low in the experimental cohort (FIG. 4). No significant main effects on the frequency, latency, or duration of pinning were observed.

Treatment effects are summarized in Table 6.

TABLE 6
Summary of the effect of CBD upon juvenile social play behaviour

| | | phenotype | risperidone | CBD | | |
|---|---|---|---|---|---|---|
| Behaviour | | evident | 0.125 mg/kg | 10 mg/kg | 30 mg/kg | 60 mg/kg |
| Social | Latency (sec) | X | X | X | X | X |
| Play | Frequency | X | X | X | X | X |
| | Duration (sec) | ✓ | X | X | X | ✓ |
| Mount | Latency (sec) | ✓ | ✓ | ✓ | ✓ | ✓ |
| | Frequency | ✓ | X | X | X | ✓ |
| | Duration (sec) | ✓ | X | X | X | ✓ |
| Pin | Latency (sec) | Very low level of pinning behaviour was observed | | | | |
| | Frequency | | | | | |
| | Duration (sec) | | | | | |

X = no effect;
✓ = significant effect

### Juvenile Irritability-Like Behaviour: Bottle Brush Task—(P30)

### Aggressive Responses

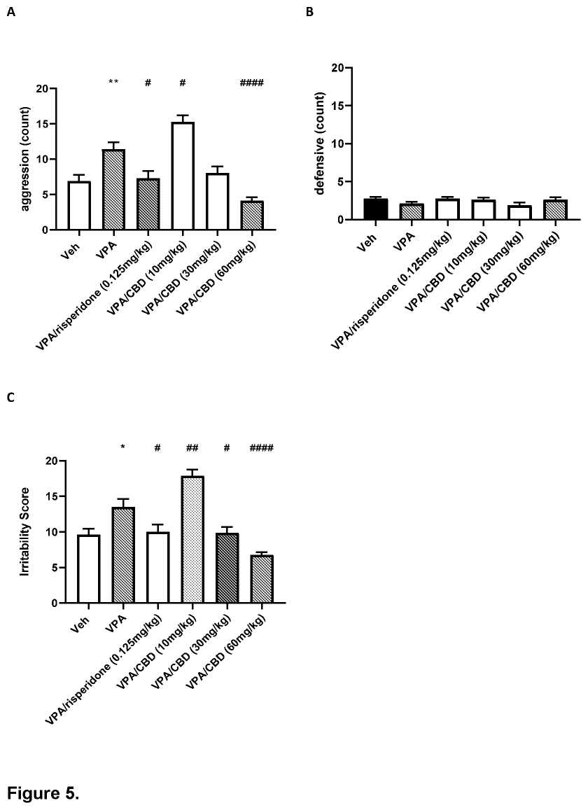

There was a significant main effect of treatment on aggressive responses (FIG. 5A F[5, 42]=18.4; P<0.0001). Further investigation revealed significant increases in aggressive responses in VPA treated rats compared to vehicle control (VEH/VEH vs VPA/VEH, P=0.0059). CBD at 10 and 60 mg/kg relative to vehicle significantly reduced the increase in aggressive responses seen in the rat VPA model (FIG. 5A, P=0.0225 and P<0.0001, respectively). Risperidone (P=0.0133) also significantly reduced the increase in aggressive responses seen in the VPA model (FIG. 5A).

### Irritability Score

There was a significant main effect on irritability (combined aggressive and defensive behaviours) (FIG. 5C, F[5, 42]=19.53; P<0.0001). Further investigation revealed significant increases in irritability score in VPA treated rats compared to vehicle control (VEH/VEH vs VPA/VEH, P=0.0162). CBD at 10, 30 and 60 mg/kg relative to vehicle significantly reduced the increase in aggressive responses seen in the rat VPA model (FIG. 5C, P=0.0052, 0.0278 and <0.0001 vs VPA/VEH, respectively). Risperidone (P=0.0361) also significantly reduced the increase in irritability score seen in the VPA model (FIG. 5C).

### Open Field Behaviour—(P31-32)

### Hyperactivity

All groups of animals were evaluated in the open field over two days following 8 and 9 days treatment with CBD (10, 30 and 60 mg/kg, p.o.), vehicle or risperidone. Thus, day 1 represents spontaneous locomotor activity and day 2 represents habituated locomotor activity.

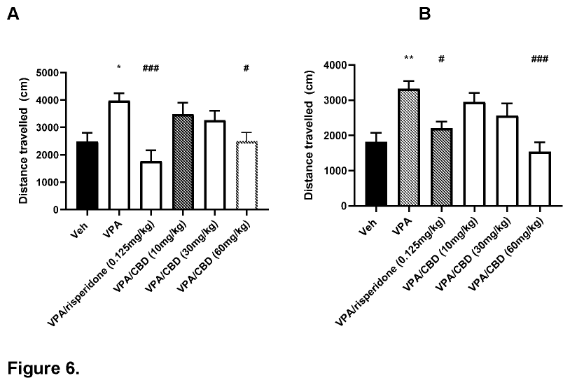

On day one, there was a significant main effect of treatment on distance travelled (FIG. 6A, F[5, 42]=5.307; P=0.0007). Specifically, there was a significant increase in locomotor activity in VPA-treated animals as compared to vehicle controls (P=0.0222 VPA/VEH vs VEH/VEH). CBD at 60 mg/kg, significantly prevented the increase in locomotor activity in VPA rats (P=0.0232 VPA/VEH vs 60 mg/kg CBD). Risperidone likewise significantly reduced locomotor hyperactivity on day one (P=0.0003).

On day two, distance travelled was also significantly altered (FIG. 6B, F[5, 42]=6.65; P=0.0001). Specifically, locomotor activity was significantly increased in VPA exposed animals as compared to vehicle controls (P=0.0011, VPA/VEH vs VEH/VEH). CBD at 60 mg/kg significantly prevented the increase in locomotor activity in VPA rats (P=0.0001 VPA/VEH vs 60 mg/kg CBD). Risperidone significantly reduced the increased locomotor activity on day two (P=0.0214).

### Circling and Grooming Behaviour

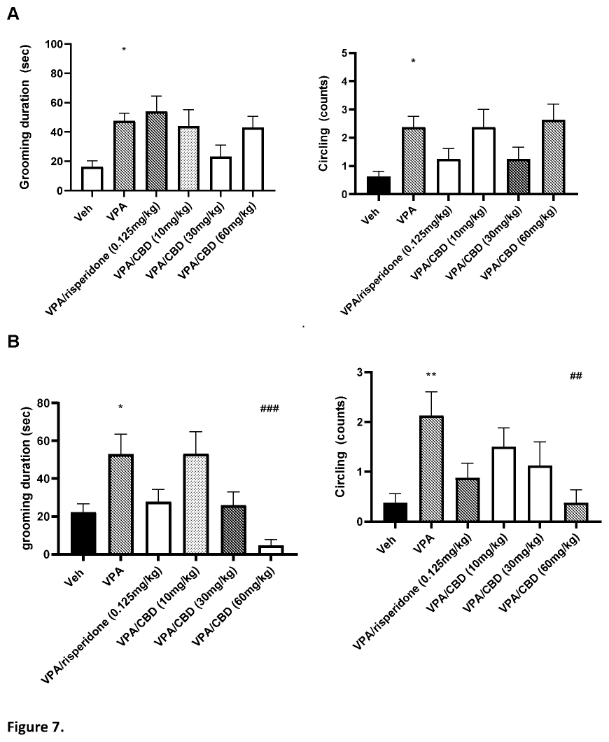

On day one there was a significant main effect on circling behaviour (F[5, 42]=3.347; P=0.0124) and self-grooming (F[5, 42]=3.315; P=0.013) (FIG. 7A). Specifically, there was a significant increase in circling and grooming behaviour in the rat VPA model relative to control (P=0.0397 and P=0.0465, respectively). CBD was without effect on this increase. On day 2, there was a significant main effect on circling behaviour (F[5, 42]=3.49; P=0.01) and self-grooming (F[5, 42]=5.711; P=0.0004) (FIG. 7B). Specifically, there was a significant increase in circling behaviour in the rat VPA model relative to control (P=0.0073), that was significantly attenuated by CBD at 60 mg/kg (P=0.0073). Likewise, a significant increase in self grooming in the rat VPA model (P=0.0435) was reversed by CBD at 60 mg/kg (P=0.0005). Risperidone activity did not reach significance on day one or two.

### Gastrointestinal Permeability—(P33)

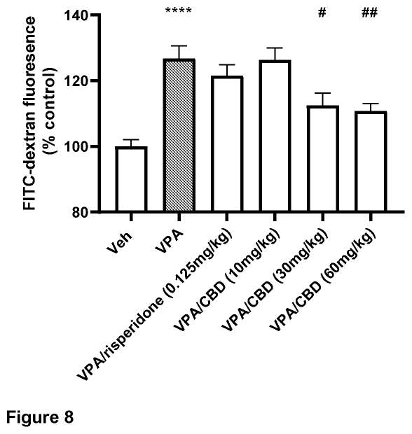

Gastrointestinal problems are widely reported in autism including constipation, diarrhoea and abdominal pain (de Theiie et al., 2011), attributed to changes in gastrointestinal microflora and inflammation. Gastrointestinal permeability can be measured employing 4 kDa FITC dextran by measuring relative fluorescence within plasma samples collected before and 4 hours after administration of CBD, vehicle or risperidone. Analysis by one-way ANOVA confirmed a significant main effect of treatment on 4 kDa dextran fluorescence (F[5, 42]=10.45; P<0.0001) (FIG. 8). Specifically, there was a significant increase in fluorescence in VPA-treated animals as compared to vehicle controls, indicative of increased gastrointestinal permeability (P<0.0001 VPA/VEH vs VEH/VEH). CBD at 30 and 60 mg/kg, significantly prevented the increase in fluorescence permeability (P=0.0168 and P=0.0061, VPA/VEH vs 30 and 60 mg/kg CBD, respectively). Risperidone was without effect (P=0.784).

### Colon Myeloperoxidase Activity—(P34)

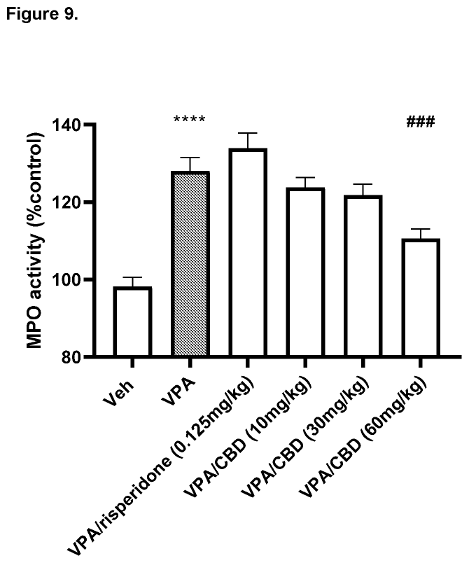

As a measure of gastrointestinal inflammation colonic myeloperoxidase (MPO) activity was determined in distal colon tissue collected 60 minutes following a final treatment with CBD, vehicle or risperidone on postnatal day 24. A significant main effect of treatment on MPO activity was revealed by one-way ANOVA (F[5, 42]=18.57; P<0.0001) (FIG. 9). Specifically, there was a significant increase MPO activity in VPA-treated animals as compared to vehicle controls (P<0.0001 VPA/VEH vs VEH/VEH). CBD at 60 mg/kg, significantly prevented the increase in MPO activity (P=0.0009, VPA/VEH vs 60 mg/kg CBD). Risperidone was without effect (P=0.6038).

### Muscle Thickness in Rat Colonic Tissue

TABLE 7

| | In utero | | Average Muscle |
|---|---|---|---|
| Group | treatment | TC treatment | Thickness (μm) |
| 1 | Vehicle | TC vehicle | 229.1 |
| 2 | VPA | TC vehicle | 186.8 |
| 3 | VPA | CBD 30 mg/kg | 220.4 |
| 4 | VPA | CBD 60 mg/kg | 207.2 |

The valproic acid induced rat model of autism results in a significant thinning of colonic muscle, as demonstrated by comparison between tissue samples from VPA-exposed versus vehicle-exposed animals. The study shows that this thinning is attenuated by treatment with CBD.

### Study Two—CBD and THC Combination

In the second study the sub-optimal and effective dose of CBD (30 and 60 mg/kg, p.o., respectively) were again tested and comparison made to treatment with CBD at the sub-optimal 30 mg/kg dose in combination with increasing dose of THC (0.75, 1.5 and 3 mg/kg, p.o.). With vehicle controls as per study one, these animals were evaluated in the same battery of behavioural and gastrointestinal readouts.

### Social Play Behaviours—(P30)

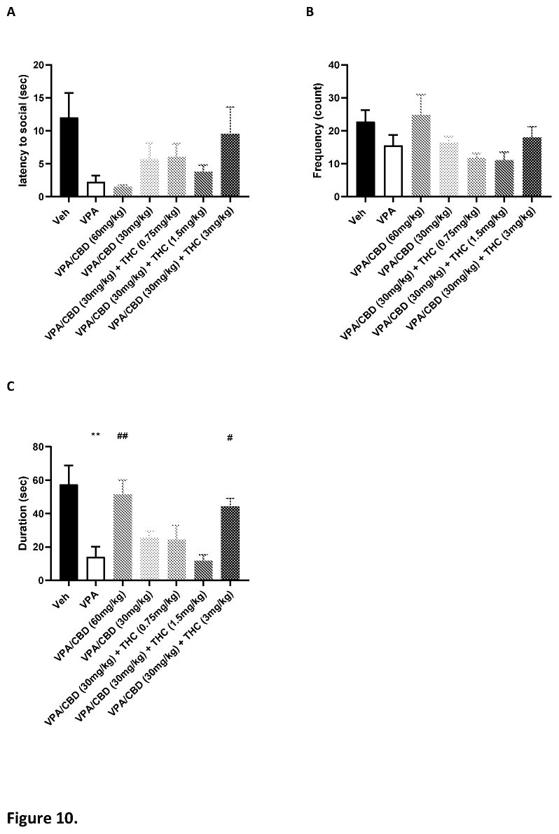

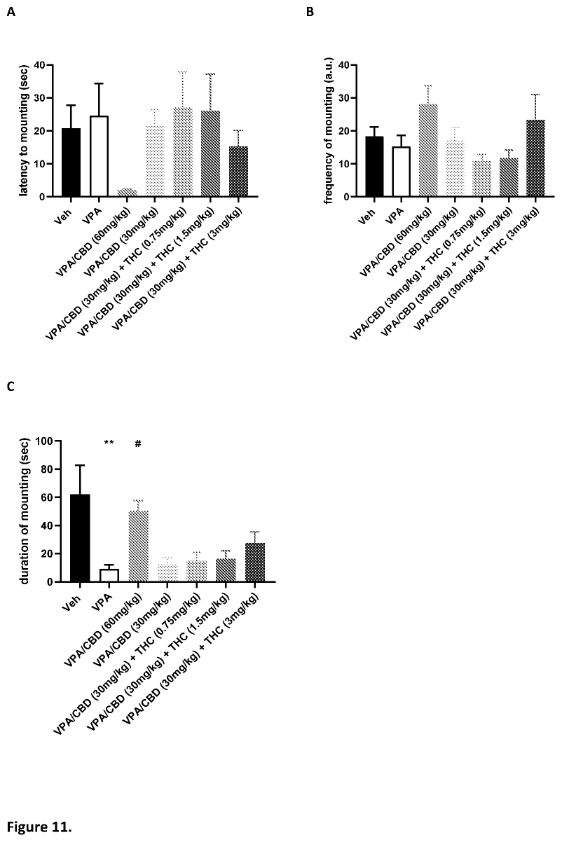

When investigating reciprocal social behaviour, one-way ANOVAs revealed no significant main effect of treatment on latency to or frequency of social play (F[6, 21]=2.372 and 2.248, P=0.066 and 0.0784, respectively) (FIGS. 10A and 11B), however there was a significant main effect on duration of social play (F[5, 18]=6.453, P=0.0006) (FIG. 10C). Post-hoc analysis revealed significant decreases in duration of social behaviour of VPA exposed animals when compared to vehicle treated control animals (VEH/VEH vs VPA/VEH, P=0.002). CBD at 60 mg/kg (P=0.0044) significantly attenuated the reduction in social play duration as per study one. The sub-optimal 30 mg/kg CBD dose was without effect. However, the sub-optimal CBD dose in combination with THC at the highest ratio (CBD 30 mg/kg+THC 3 mg/kg) significantly attenuated the reduction in social play duration (P=0.0085).

When investigating mounting behaviours, one-way ANOVA showed there was no significant main effect of treatment on latency to engage in mounting play behaviour (F[6, 21]=1.218, P=0.3361) (FIG. 11A) or frequency of mounting behaviour (F[6, 21]=1.893, P=0.1295) (FIG. 11B). However, one-way ANOVA revealed a significant main effect on the duration of mounting play (F[6, 21]=4.684, P=0.0036) (FIG. 11C). Post-hoc analysis of duration of mounting behaviour revealed a significant effect of valproic acid exposure when compared to vehicle controls (P=0.0046) a decrease in play reversed following treatment with CBD at 60 mg/kg (P=0.0364). Combination of the sub-optimal 30 mg/kg CBD dose with increasing THC was without effect on duration of mounting play behaviour.

Risperidone (0.125 mg/kg) was without effect on any other measure of play or social interaction in juvenile animals as per previous experiments.

Treatment effects are summarized in Table 8.

TABLE 8
Summary of the effect of CBD alone and in combination
with THC upon juvenile social play behaviour

| | | phenotype | CBD alone | | CBD 30 mg/kg plus THC | | |
|---|---|---|---|---|---|---|---|
| Behaviour | | evident | 30 mg/kg | 60 mg/kg | 0.75 mg/kg | 1.5 mg/kg | 3 mg/kg |
| Social | Latency (sec) | X | X | X | X | X | X |
| Play | Frequency | X | X | X | X | X | X |
| | Duration (sec) | ✓ | X | ✓ | X | | ✓ |
| Mount | Latency (sec) | X | X | X | X | X | X |
| | Frequency | X | X | X | X | X | X |
| | Duration (sec) | ✓ | X | ✓ | X | X | X |
| Pin | Latency (sec) | Very low level of pinning behaviour was observed | | | | | |
| | Frequency | | | | | | |
| | Duration (sec) | | | | | | |

X = no effect;
✓ = significant effect

### Juvenile Irritability-Like Behaviour: Bottle Brush Task—(P30)

### Aggressive Responses

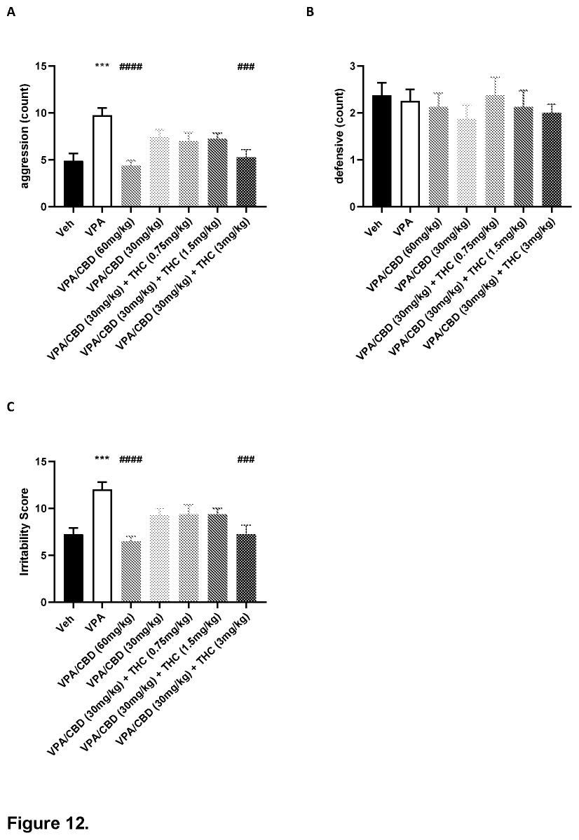

There was a significant main effect of treatment on aggressive responses (FIG. 12A F[6, 49]=6.102; P<0.0001). Further investigation revealed significant increases in aggressive responses in VPA treated rats compared to vehicle control (VEH/VEH vs VPA/VEH, P=0.0002). CBD at 60 mg/kg relative to vehicle significantly reduced the increase in aggressive responses seen in the rat VPA model as per study one (P<0.0001). The sub-optimal 30 mg/kg CBD dose was without effect. However, the sub-optimal CBD dose in combination with THC at the highest ratio (CBD 30 mg/kg+THC 3 mg/kg) significantly attenuated the reduction in social play duration (P=0.0006).

### Irritability Score

There was a significant main effect on irritability (combined aggressive and defensive behaviours) (FIG. 12C, F[6, 49]=6.001; P<0.0001). Further investigation revealed significant increases in irritability score in VPA treated rats compared to vehicle control (VEH/VEH vs VPA/VEH, P=0.0004). CBD at 60 mg/kg relative to vehicle significantly reduced the increase in aggressive responses seen in the rat VPA model (P<0.0001 vs VPA/VEH). The sub-optimal 30 mg/kg CBD dose was without effect. However, the sub-optimal CBD dose in combination with THC at the highest ratio (CBD 30 mg/kg+THC 3 mg/kg) significantly attenuated the reduction in social play duration (P=0.0004).

### Open Field Behaviour—(P31-32)

### Hyperactivity

All groups of animals were evaluated in the open field over two days following 8 and 9 days treatment with CBD alone (30 and 60 mg/kg, p.o.), CBD in combination with increasing THC dose (0.75, 1.5 and 3 mg/kg, p.o.), or vehicle. Thus, day 1 represents spontaneous locomotor activity and day 2 represents habituated locomotor activity.

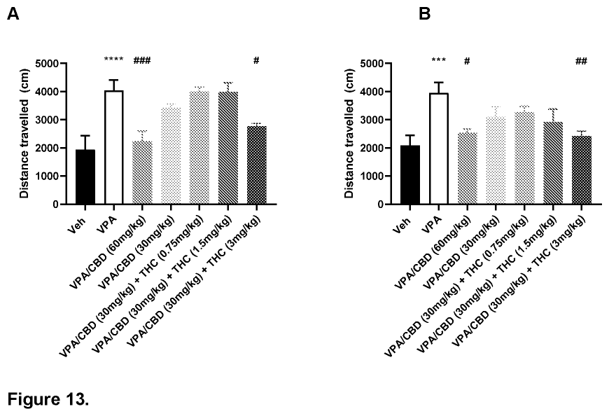

On day one, there was a significant main effect of treatment on distance travelled (FIG. 13A, F[6, 49]=8.289; P<0.0001). Specifically, there was a significant increase in locomotor activity in VPA-treated animals as compared to vehicle controls (P<0.0001 VPA/VEH vs VEH/VEH). CBD at 60 mg/kg, significantly prevented the increase in locomotor activity in VPA rats (P=0.0009 VPA/VEH vs 60 mg/kg CBD). The sub-optimal 30 mg/kg CBD dose was without effect. However, the sub-optimal CBD dose in combination with THC at the highest ratio (CBD 30 mg/kg+THC 3 mg/kg) significantly attenuated the increase in day one hyperactivity (P=0.0321).

On day two, distance travelled was also significantly altered (FIG. 13B, F[6, 49]=3.8; P=0.0035). Specifically, locomotor activity was significantly increased in VPA exposed animals as compared to vehicle controls (P=0.0008, VPA/VEH vs VEH/VEH). CBD at 60 mg/kg significantly prevented the increase in locomotor activity in VPA rats (P=0.0167 VPA/VEH vs 60 mg/kg CBD). The sub-optimal 30 mg/kg CBD dose was without effect. However, the sub-optimal CBD dose in combination with THC at the highest ratio (CBD 30 mg/kg+THC 3 mg/kg) significantly attenuated the day 2 hyperactivity and failed habituation of the VPA autism model (P=0.0082).

### Circling and Grooming Behaviour

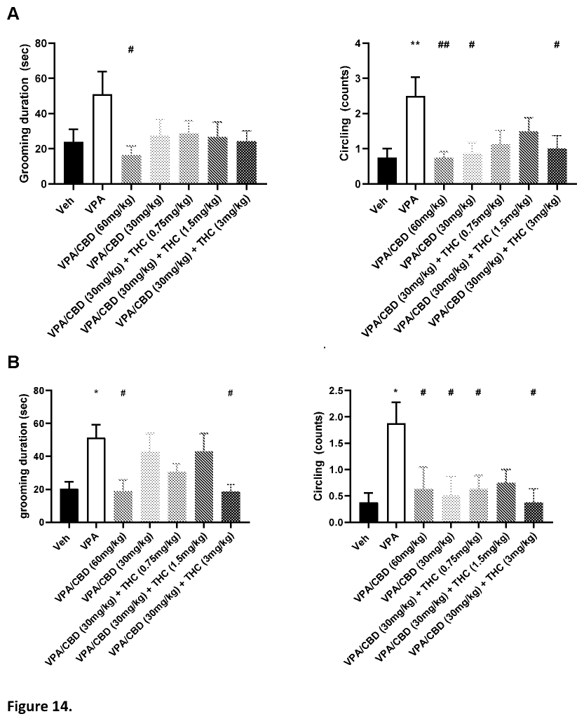

On day one there was a significant main effect on circling behaviour (F[6, 49]=3.006; P=0.014) but not self-grooming (F[6, 49]=1.681; P=0.1457) (FIG. 14A). Specifically, there was a significant increase in circling behaviour in the rat VPA model relative to control (P=0.0072). CBD at 30 and 60 mg/kg, significantly prevented the increase in circling in VPA rats (P=0.0072 and P=0.0147 VPA/VEH vs 30 and 60 mg/kg CBD, respectively). Moreover, the sub-optimal CBD dose in combination with 3 mg/kg THC also attenuated this increase in stereotypical behaviour. There was a post hoc significant effect of 60 mg/kg CBD in reducing self-grooming as compared to VPA rats (P=0.0291 VPA/VEH vs 60 mg/kg CBD), but no other treatment effects reached significance.

On day 2, there was a significant main effect on circling behaviour (F[6, 49]=2.684; P=0.0248) and self-grooming (F[6, 49]=3.161; P=0.0106) (FIG. 14B). Specifically, there was a significant increase in circling behaviour in the rat VPA model relative to control (P=0.0073), that was significantly attenuated by CBD at 60 mg/kg (P=0.0073). The sub-optimal 30 mg/kg CBD dose was without effect. However, the sub-optimal CBD dose in combination with THC at 0.75 and 3 mg/kg significantly attenuated this VPA-induced increase in stereotypy. Likewise, a significant increase in self grooming in the rat VPA model as compared to vehicle control (P=0.035) was reversed by CBD at 60 mg/kg (P=0.0251). The sub-optimal 30 mg/kg CBD dose was without effect. However, the sub-optimal CBD dose in combination with THC at 3 mg/kg significantly attenuated this VPA-induced increase.

### Gastrointestinal Permeability—(P33)

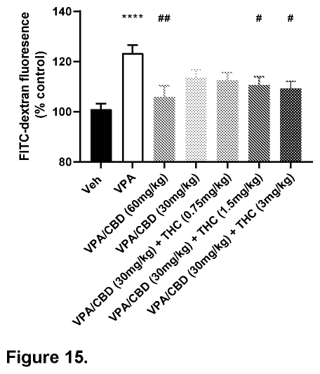

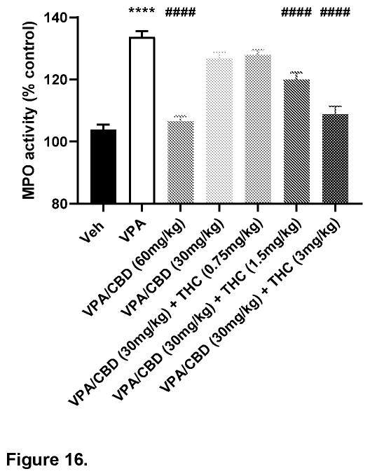

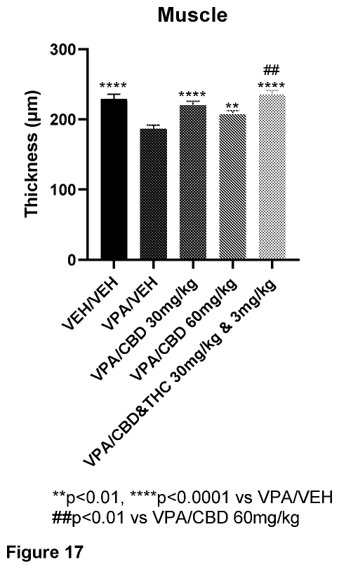

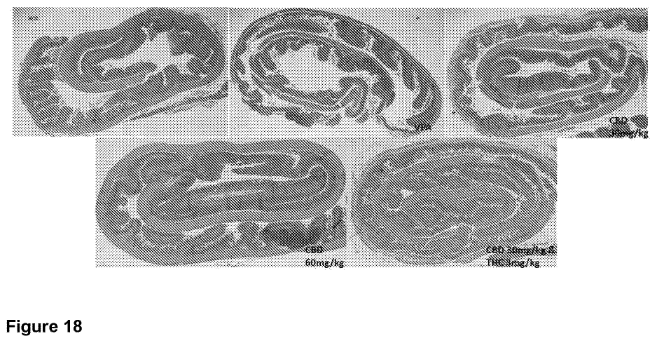

Gastrointestinal problems are widely reported in autism including constipation, diarrhoea and abdominal pain (de Theije et al., 2011), attributed to changes in gastrointestinal microflora, inflammation and increased gastrointestinal permeability (de Magistris et al 2010). Gastrointestinal permeability can be measured employing 4 kDa FITC dextran by measuring relative fluorescence within plasma samples collected before and 4 hours after administration of CBD alone or in combination with THC or vehicle. Analysis by one-way ANOVA confirmed a significant main effect of treatment on 4 kDa dextran fluorescence (F[6, 49]=4.877; P=0.0006) (FIG. 15). Specifically, there was a significant increase in fluorescence in VPA-treated animals as compared to vehicle controls, indicative of increase gastrointestinal permeability (P<0.0001 VPA/VEH vs VEH/VEH). CBD at 60 mg/kg, significantly prevented the increase in fluorescence permeability (P=0.0018, VPA/VEH vs 60 mg/kg CBD, respectively). While the sub-optimal 30 mg/kg CBD dose was without effect, the sub-optimal CBD dose in combination with THC at 1.5 and 3 mg/kg significantly attenuated this VPA-induced increase (P=0.0394 and P=0.0165, respectively).

### Colon Myeloperoxidase Activity—(P34)

As a measure of gastrointestinal inflammation colonic myeloperoxidase (MPO) activity was determined in distal colon tissue collected 60 minutes following a final treatment with CBD, vehicle or risperidone on postnatal day 24. A significant main effect of treatment on MPO activity was revealed by one-way ANOVA (F[6, 49]=37.68; P<0.0001) (FIG. 16). Specifically, there was a significant increase MPO activity in VPA-treated animals as compared to vehicle controls (P<0.0001 VPA/VEH vs VEH/VEH). CBD at 60 mg/kg, significantly prevented the increase in MPO activity (P<0.0001, VPA/VEH vs 60 mg/kg CBD). While the sub-optimal dose of CBD (30 mg/kg) was without significant effect, treatment of the sub-optimal CBD dose in combination with THC at 1.5 and 3 mg/kg significantly reduced the VPA-induced increase in MPO activity.

### Muscle Thickness

TABLE 9

| | In utero | | Average Muscle |
|---|---|---|---|
| Group | treatment | TC treatment | Thickness (μm) |
| 1 | Vehicle | TC vehicle | 229.1 |
| 2 | VPA | TC vehicle | 186.8 |
| 3 | VPA | CBD 30 mg/kg | 220.4 |
| 4 | VPA | CBD 60 mg/kg | 207.2 |
| 5 | VPA | CBD 30 mg/kg & | 235.0 |
| | | THC 3 mg/kg | |

The valproic acid induced rat model of autism results in a significant thinning of colonic muscle, as demonstrated by comparison between tissue samples from VPA-exposed versus vehicle-exposed animals. The above results show that thinning is significantly attenuated by co-administration of CBD and THC, in particular at a combination of 30 mg/kg & THC 3 mg/kg.

### CONCLUSION

The results show a Pearson correlation between gastrointestinal permeability and ASD behavioural symptoms, and between MPO activity and ASD behavioural symptoms. The p values show a significant correlation between behaviour and MPO activity and between behaviour and GI permeability. The results also show that a cannabinoid, for example CBD in combination with THC, has the effect of increasing colonic muscle thickness in a rat model of autism.
Aggressive behaviour and MPO activity p=0.0001681
Aggressive behaviour and GI Permeability p=0.0133
Irritability and MPO activity p=0.00048
Irritability and GI Permeability p=0.01522

The current study identifies robust changes in social, stereotypical, exploratory and irritability behaviour that are evident in a rodent in utero VPA model of autism, relative to vehicle controls. The aforementioned behavioural changes are reduced by sub-chronic treatment with CBD alone, identifying a minimum effective dose of 60 mg/kg. Moreover, the second study first repeated these findings, and then also demonstrated a potential beneficial additive effect of combination of sub-optimal CBD dose (30 mg/kg) and increasing THC dose (0.75, 1.5 and 30 mg/kg).

### 3 REFERENCES

de Magistris L, Familiari V, Pascotto A, Sapone A, Frolli A, lardino P, Carteni M, De Rosa M, Francavilla R, Riegler G, Militerni R, Bravaccio C. Alterations of the intestinal barrier in patients with autism spectrum disorders and in their first-degree relatives. J Pediatr Gastroenterol Nutr. 2010 October; 51 (4): 418-24.
de Theije C G, Wu J, da Silva S L, Kamphuis P J, Garssen J, Korte S M, Kraneveld A D. Pathways underlying the gut-to-brain connection in autism spectrum disorders as future targets for disease management. Eur J Pharmacol. 2011 668 Suppl 1: S70-80.
Ingram J L, Peckham S M, Tisdale B, Rodier P M. Prenatal exposure of rats to valproic acid reproduces the cerebellar anomalies associated with autism. Neurotoxicol Teratol. 2000 22 (3): 319-24.
Kononoff, J, Melas P A, Kallupi M, de Guglielmo G, Kimbrough A, Scherma M, Fadda P, Kandel D B, Kandel E R, George O. Adolescent cannabinoid exposure induces irritability-like behavior and cocaine cross-sensitization without affecting the escalation of cocaine self-administration in adulthood. Sci Rep. 2018 8:13893.
Pellis S M, Pellis V C. The playful brain: venturing to the limits of neuroscience. Oneworld Publications; Oxford: 2009.
Pellis S M, Field E F, Smith L K, Pellis V C. Multiple differences in the play fighting of male and female rats. Implications for the causes and functions of play. Neurosci Biobehav Rev. 1997 21 (1): 105-20. Review.
Riittinen M L, Lindroos F, Kimanen A, Pieninkeroinen E, Pieninkeroinen I, Sippola J, Veilahti J, Bergström M, Johansson G. Impoverished rearing conditions increase stress-induced irritability in mice. Dev Psychobiol. 1986 March; 19 (2): 105-11.
Schneider T, Przewlocki R. Behavioral alterations in rats prenatally exposed to valproic acid: animal model of autism. Neuropsychopharmacology. 2005 30 (1): 80-9.
Thevaranjan N, Puchta A, Schulz C, Naidoo A, Szamosi J C, Verschoor C P, Loukov D, Schenck L P, Jury J, Foley K P, Schertzer J D, Larché MJ, Davidson D J, Verdú E F, Surette M G, Bowdish D M E. Age-Associated Microbial Dysbiosis Promotes Intestinal Permeability, Systemic Inflammation, and Macrophage Dysfunction. Cell Host Microbe. 2017 21 (4): 455-66.
Vanderschuren L J, Niesink R J, Van Ree J M. The neurobiology of social play behavior in rats. Neurosci Biobehav Rev. 1997 21 (3): 309-26. Review.
Wagner G C, Reuhl K R, Cheh M, McRae P, Halladay A K. A new neurobehavioral model of autism in mice: pre- and postnatal exposure to sodium valproate. J Autism Dev Disord. 2006 36 (6): 779-93.

### Aspects and Embodiments

1. A cannabinoid for use in the prevention or treatment of gastrointestinal hyperpermeability in a subject.
2. A cannabinoid for use according to paragraph 1 wherein the subject has been diagnosed with an autism spectrum disorder, or is susceptible to an autism spectrum disorder.
3. The use according to paragraph 1 or paragraph 2 wherein the subject has been diagnosed with, or is susceptible to Crohn's disease, celiac disease, ulcerative colitis, diabetes type 1, arthritis including spondylarthrites and psoriatic arthritis, inflammation, for example gastrointestinal inflammation.
4. A cannabinoid for use in the prevention or treatment of a symptom of an autism spectrum disorder in a subject, wherein the subject has been diagnosed as having gastrointestinal hyperpermeability or diagnosed as being at risk or susceptible to gastrointestinal hyperpermeability.
5. The use according to paragraph 4 wherein the subject has one or more symptoms of autism spectrum disorder, optionally wherein the symptom is a behavioural symptom or a gastrointestinal disorder, or a combination thereof.
6. A cannabinoid for use according to paragraph 5 wherein the subject has been diagnosed with an autism spectrum disorder.
7. A cannabinoid for use in the prevention or reduction in gastrointestinal inflammation in a subject.
8. A cannabinoid for according to paragraph 7 wherein the subject has been diagnosed with, or is susceptible to autism spectrum disorder, crohn's disease, celiac disease, ulcerative colitis, diabetes type 1, arthritis including spondylarthrites and psoriatic arthritis, inflammation in particular gastrointestinal inflammation.
9. The use according to paragraph 8 or 9 wherein the subject has or is susceptible to gastrointestinal hyperpermeability.
10. A cannabinoid for use in the prevention or treatment of a symptom of an autism spectrum disorder in a subject, by preventing or reducing gastrointestinal permeability.
11. A cannabinoid for use according to paragraph 10 wherein the subject has been diagnosed with an autism spectrum disorder.
12. A cannabinoid for use according to paragraph 10 wherein the the symptom of an autism spectrum disorder is a behavioural symptom and/or a a gastrointestinal symptom.
13. A cannabinoid for use in the prevention or reduction in gastrointestinal inflammation in a subject, by preventing or reducing gastrointestinal permeability.
14. A cannabinoid for use according to paragraph 13, wherein the subject is susceptible to, or has, autism spectrum disorder, Crohn's disease, celiac disease, ulcerative colitis, diabetes type 1, arthritis including spondylarthrites and psoriatic arthritis, or inflammation in particular gastrointestinal inflammation.
15. Use according to any one of the preceding paragraphs wherein the cannabinoid is cbd, THC or a combination of CBD and THC.
16. The use according to any one of the preceding paragraphs, wherein the cannabinoid is suitable for oral administration.
17. The use according to any one of the preceding paragraphs wherein the cannabinoid is in the form of a pharmaceutical or nutritional composition.
18. The use according any one of the preceding paragraphs wherein the cannabinoid is administered to a subject in need thereof, preferably wherein the subject is human, at a dose of 0.1 to 20 mg/kg, suitably 0.5 to 15 mg/kg, suitably 0.8 to 12 mg/kg CBD and/or 0.05 to 6 mg/kg, suitably 0.1 to 5 mg/kg, most suitably 0.1 to 4 mg/kg body weight THC.
19. A dosage form of a cannabinoid comprising 0.1 to 20 mg/kg body weight of CBD and/or 0.05 to 6 mg/kg mg/kg body weight THC.
20. A cannabinoid for use in the prevention or treatment of a symptom of ASD in a subject, by preventing or reducing colonic muscle thinning.
21. A method of preventing or treating a symptom of ASD in a subject, wherein the method comprises administering a cannabinoid to the subject, wherein the cannabinoid prevents or reduces colonic muscle thinning.
22. A cannabinoid for use in the prevention or treatment of a symptom of ASD in a subject, wherein the subject has, or has been tested as having, colonic muscle thinning.
23. A method of preventing or treating a symptom of ASD in a subject, wherein the method comprises administering a cannabinoid to the subject, wherein the subject has, or has been tested as having, gastrointestinal hyperpermeability or gastrointestinal inflammation.
24. A cannabinoid for use according to claim 20 or 22, or a method according to paragraphs 21 or 23 wherein the cannabinoid may prevent or reduce colonic muscle thinning or increase colonic muscle thickness, and/or maintain colonic muscle thickness.
25. A cannabinoid for use according to paragraphs 20, 22 or 24, wherein the symptom of ASD includes a behavioural symptom.
26. A cannabinoid for use according to paragraphs 20, 22, 24 or 25 or a method according to claim 19, 23, 24 or 25 wherein a subject may have been diagnosed with ASD or a symptom thereof.
27. A method according to paragraphs 19, 23, 24 or 25 wherein the method comprises a step of diagnosing a subject with one or more of ASD or a symptom thereof, Crohn's disease, celiac disease, ulcerative colitis, diabetes type 1, arthritis including spondyloarthritis and psoriatic arthritis, or inflammation, in particular gastrointestinal inflammation.
28. A cannabinoid for use according to paragraphs 20, 22, 24 or 25 or a method according to claim 19, 23, 24 or 25 wherein the cannabinoid is CBD, tetrahydrocannabinol (THC), or a combination of CBD and THC.

1. A method of preventing or treating a subject having symptoms associated with autism spectrum disorder, wherein i) the subject has been diagnosed as having or being at risk of having gastrointestinal hyperpermeability, and/or ii) wherein the subject has been diagnosed as having or at risk of having colonic muscle thinning, wherein the method comprises administering a cannabinoid to said subject.

2. The method according to claim 1, wherein the administering step prevents or reduces gastrointestinal hyperpermeability and/or colonic muscle thinning.

3. The method according to claim 1, wherein the symptom of an autism spectrum disorder is a behavioural symptom and/or a gastrointestinal symptom.

4. The method according to claim 1, wherein the subject has one or more symptoms of autism spectrum disorder, optionally wherein the one or more symptoms are behavioural and/or gastrointestinal symptoms.

5. The method according to claim 1, wherein the subject has been diagnosed as having an autism spectrum disorder, or is susceptible to an autism spectrum disorder or a symptom thereof.

6. The method according to claim 2, wherein the cannabinoid prevents or reduces colonic muscle thinning, increases colonic muscle thickness, and/or maintains colonic muscle thickness.

7. The method according to claim 1, wherein the subject has been diagnosed with, or is susceptible to Crohn's disease, celiac disease, ulcerative colitis, diabetes type 1, arthritis including spondylarthrites and psoriatic arthritis, inflammation, for example gastrointestinal inflammation.

8-16. (canceled)

17. The method according to claim 1, wherein the cannabinoid is CBD or a combination of CBD and tetrahydrocannabinol (THC).

18. The method according to claim 1, wherein the cannabinoid is suitable for oral administration.

19. The method according to claim 1, wherein the cannabinoid is in the form of a pharmaceutical or nutritional composition.

20. The method according to claim 1, wherein the cannabinoid is administered to a subject in need thereof at a dose of 0.1 to 20 mg/kg, suitably 0.5 to 15 mg/kg, suitably 0.8 to 12 mg/kg body weight CBD; optionally in combination with 0.05 to 6 mg/kg, suitably 0.1 to 5 mg/kg, most suitably 0.1 to 4 mg/kg body weight THC.

21. The method according to claim 1, wherein the cannabinoid is administered to a subject in need thereof at a dose of 0.1 to 20 mg/kg body weight of body weight CBD, optionally in combination with 0.05 to 6 mg/kg body weight THC.

22. The method according to any of the preceding claims wherein the subject is human, suitably a child.

23. (canceled)
